(19) United States
(12) Patent Application Publication
(10) Pub. No.: US 2021/0275440 A1
(43) Pub. Date: Sep. 9, 2021

(54) MICROBEAD COMPOSITIONS AND METHODS FOR DELIVERING AN AGENT

(71) Applicant: THE UNIVERSITY OF MEMPHIS RESEARCH FOUNDATION, Memphis, TN (US)

(72) Inventors: ANKITA MOHAPATRA, MEMPHIS, TN (US); MICHAEL A. HARRIS, MEMPHIS, TN (US); BASHIR I. MORSHED, MEMPHIS, TN (US); JESSICA A. JENNINGS, MEMPHIS, TN (US); JOEL BUMGARDNER, MEMPHIS, TN (US); TOMOKO FUJIWARA, MEMPHIS, TN (US); SANJAY R. MISHRA, MEMPHIS, TN (US); DAVID A. LEVINE, MEMPHIS, TN (US); GREGORY MCGRAW, MEMPHIS, TN (US); WARREN O. HAGGARD, MEMPHIS, TN (US)

(73) Assignee: THE UNIVERSITY OF MEMPHIS RESEARCH FOUNDATION, MEMPHIS, TN (US)

(21) Appl. No.: 16/334,542

(22) PCT Filed: Sep. 27, 2017
(86) PCT No.: PCT/US2017/053688

§ 371 Date: Mar. 19, 2019

Related U.S. Application Data
(60) Provisional application No. 62/401,751, filed on Sep. 29, 2016.

(51) Int. Cl. A61K 9/00; A61K 38/14; A61K 41/00; A61K 9/16

(52) U.S. Cl. CPC A61K 9/0009; A61K 38/14; A61K 9/1682; A61K 9/1652; A61K 41/0028

(57) ABSTRACT

The invention provides microbeads comprising chitosan, a magnetic nanoparticle, and an agent, and methods for using such microbeads for the local delivery of biologically active agents to an open fracture, complex wound or other site of infection or disease.

### CROSS-REFERENCE TO RELATED APPLICATIONS

This application claims the benefit of and priority to U.S. Provisional Patent Application Ser. No. 62/401,751 filed Sep. 29, 2016, which is incorporated herein by reference in its entirety.

### BACKGROUND OF THE INVENTION

Traumatic injuries are devastating and their infections can be difficult to treat, often resulting in multiple surgeries and increased costs. Infections can result in high healthcare costs, high mortality rates, and significantly higher amputation rates than those from bacterial infections alone. A limitation of current local therapeutic agent delivery systems is that release is poorly controlled, often resulting in burst release of large amounts of drug followed by sub-therapeutic levels afterward. After releasing drugs, many local delivery systems must then be retrieved, which requires invasive surgical procedures.

Because current methods for treating or preventing infection are inadequate, improved compositions and methods for providing agents to prevent or treat an infection at a site of trauma are urgently required.

### SUMMARY OF THE INVENTION

As described below, the present invention features compositions comprising chitosan microbeads that provide for the delivery of therapeutic agents, which can be triggered non-invasively for improved control over drug availability.

In one aspect, the invention provides a microbead containing cross-linked chitosan, a magnetic nanoparticle, and an agent.

In another aspect, the invention provides a method for producing a chitosan microbead, the method involving dissolving chitosan in an acidic solution; adding magnetic nanoparticles and an agent to the solution; providing a mixture of surfactant, oil, and a polymer; and adding the chitosan solution to the oil and incubating until beads form. In one embodiment, the method further involves incorporating an effective amount of one or more agents into the solution.

In another aspect, the invention provides a microbead generated according to the method of a previous aspect.

In another aspect, the invention provides a method for treating or preventing an infection in a subject at a site of trauma, the method involving contacting the site with a chitosan microbead of any previous aspect and applying an external stimulus. In one embodiment, the trauma is selected from a fracture, open fracture, wound, complex wound, or surgical site.

In another aspect, the invention provides a method for the local and temporally controlled delivery of an agent to a site, the method involving contacting the site with a chitosan microbead containing an agent and applying an external stimulus at a desired time point, thereby temporally controlling delivery of the agent to the site.

In another aspect, the invention provides a kit containing a chitosan microbead of any previous aspect for use in treating a trauma site or delivering an agent.

In various embodiments of any of the above aspects or any other aspect of the invention delineated herein, the chitosan is cross-linked to a polymer. In various embodiments of any of the above aspects, the polymer is polyethylene dimethacrylate (PEGDMA) In various embodiments of any of the above aspects, the microbead contains an effective amount of an agent that is any one or more of a polypeptide, polynucleotide or small compound. In various embodiments of any of the above aspects, the agent is an analgesic, angiogenic agent, antimicrobial, antibody, antifungal, anti-inflammatory, anti-thrombotic, chemotherapeutic, growth factor, hormone, or steroid agent. In various embodiments of any of the above aspects, the antimicrobial agent is selected from the group consisting of antifungal, antibacterial, and antiviral agents. In various embodiments of any of the above aspects, the antimicrobial agents are amphotericin B, vancomycin, and/or amikacin. In various embodiments of any of the above aspects, the effective amount of the agent is sufficient to reduce the survival or proliferation of a fungal cell (e.g., Candida albicans) or bacterial cell (Pseudomonas aeruginosa (lux) or Staphylococcus aureus). In various embodiments of any of the above aspects, the composition releases at least about 0.2-50 μg of an antimicrobial agent per hour. In various embodiments of any of the above aspects, the microbead is biodegradable over at least about one, two, three, four, or five days, or one, two, three, or four weeks. In various embodiments of any of the above aspects, the agent is any one or more of an analgesic, angiogenic agent, antimicrobial, antibody, antifungal, anti-inflammatory, anti-thrombotic, chemotherapeutic, growth factor, hormone, or steroid agent. In various embodiments of any of the above aspects, the microbead releases about 2 μg-1000 mg of the agent in 1-72 hours. In various embodiments of any of the above aspects, the stimulus is a magnetic field. In various embodiments of any of the above aspects, the stimulus is a electric field. In various embodiments of any of the above aspects, the stimulus is applied for 30 minutes.

The invention provides chitosan microbeads comprising a therapeutic agent and methods of using such microbeads for the local delivery of biologically active agents (e.g., antimicrobials, chemotherapeutics) to an open fracture, complex wound or other site of infection or disease. Compositions and articles defined by the invention were isolated or otherwise manufactured in connection with the examples provided below. Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “chitosan microbead” is meant a microscopic particle or sphere comprising cross-linked chitosan. In one embodiment, a microbead is at least about 0.001 um to about 5 mm in diameter.

By “chitosan” is meant a chitin-derived polymer that is at least 20% deacetylated. In various embodiments, chitosan is at least about 50% deacetylated. In particular embodiments, chitosan is at least about 61% or 71% deacetylated. Chitin is a linear polysaccharide consisting of (1-4)-linked 2-acetamido-2-deoxy-b-D-glucopyranose. Chitosan is a linear polysaccharide consisting of (1-4)-linked 2-amino-2-deoxy-b-D-glucopyranose. An exemplary chitosan polymer is shown by the formula below. In one embodiment, chitosan has a molecular weight of about 250 kD.

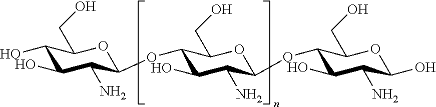

By “acid treated chitosan” is meant chitosan that is solubilized in an acidic solution.

By “vancomycin” is meant the compound (1S,2R,18R,19R,22S,25R,28R,40S)-48-{[(2S,3R,4S,5S,6R)-3-{[(2S,4S,5S,6S)-4-amino-5-hydroxy-4,6-dimethyloxan-2-yl]oxy}-4,5-dihydroxy-6-(hydroxymethyl)oxan-2-yl]oxy}-22-(carbamoylmethyl)-5,15-dichloro-2,18,32,35,37-pentahydroxy-19-[(2R)-4-methyl-2-(methylamino)pentanamido]-20,23,26,42,44-pentaoxo-7,13-dioxa-21,24,27,41,43-pentaazaoctacyclo[26.14.2.2^{3,6}.2^{14,17}.1^{8,12}.1^{29,33}.0^{10,25}.0^{34,39}]pentaconta-3,5,8(48),9,11,14,16,29(45),30,32,34,36,38,46,49-pentadecaene-40-carboxylic acid and CAS number 1404-90-6. Vanomycin is shown by the formula below.

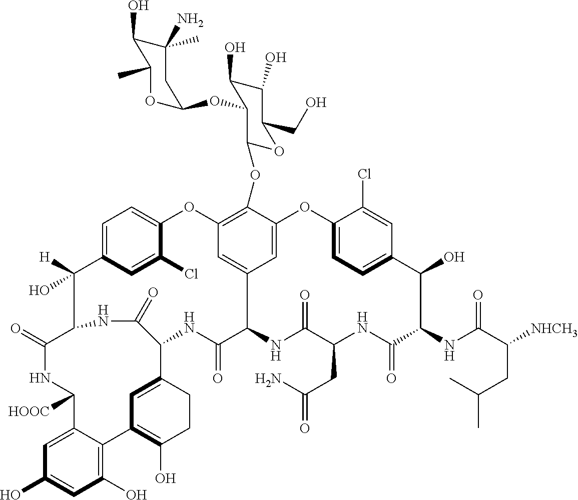

By “polyethylene glycol (PEG)” is meant an oligomer or polymer of ethylene oxide. Commercially available PEG ranges in molecular weight from 300 g/mol to 10,000,000 g/mol. An exemplary PEG is shown by the formula below.

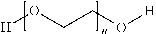

In particular embodiments, PEG molecular weight is 6000 g/mol, 8,000 g/mol, 10,000 g/mol. The degradation profile of the chitosan/PEG composition can be tailored to the desired level by increasing or decreasing the molecular weight of the PEG. In particular, when lower molecular weight PEG is used degradation is enhanced. When higher molecular weight PEG is used degradation is decreased.

By “polyethylene dimethacrylate (PEGDMA)” is meant an oligomer or polymer of ethylene oxide with dimethacrylate to form PEGDMA. Commercially available PEGDMA ranges in molecular weight from 1 g/mol to 10,000 g/mol.

An exemplary PEGDMA is shown by the formula below:

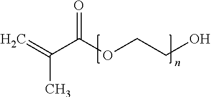

In an embodiment, PEGDMA is polyethylene glycol dimethacrylate (PEGDMA). In particular embodiments, PEGDMA molecular weight is 100 g/mol, 200 g/mol, 300 g/mol, 400 g/mol, 500 g/mol, 600 g/mol, 700 g/mol, 800 g/mol, 900 g/mol, 1,000 g/mol. In particular embodiments, PEGDMA can be a polydisperse mixture of multiple molecular weights. The degradation profile of the chitosan/PEGDMA composition can be tailored to the desired level by increasing or decreasing the molecular weight of the PEGDMA. In particular, when lower molecular weight PEGDMA is used degradation is enhanced. When higher molecular weight PEGDMA is used degradation is decreased.

By “nanoparticle” is meant a composite structure of nanoscale dimensions. In particular, nanoparticles are typically particles of a size in the range of from about 1 to about 1000 nm, and are usually spherical although different morphologies are possible depending on the nanoparticle composition. The portion of the nanoparticle contacting an environment external to the nanoparticle is generally identified as the surface of the nanoparticle. In nanoparticles herein described, the size limitation can be restricted to two dimensions and so that nanoparticles herein described include composite structure having a diameter from about 1 to about 1000 nm, where the specific diameter depends on the nanoparticle composition and on the intended use of the nanoparticle according to the experimental design. For example, nanoparticles to be used in several therapeutic applications typically have a size of about 200 nm or below, and the ones used, in particular, for delivery associated to therapeutic agents typically have a diameter from about 1 to about 100 nm.

By “degrades” is meant physically or chemically breaks down in whole or in part. Preferably, the degradation represents a physical reduction in the mass by at least about 10%, 25%, 50%, 75%, 80%, 85%, 90%, 95% or 100%.

By “long term release” is meant elution of an agent over the course of twenty-four-seventy-two hours or longer. In particular embodiments, release occurs over one, two, three or four weeks.

By “wound management device” or “wound healing device” is meant any material used to protect or promote healing at a site of trauma.

By “agent” or “therapeutic agent” is meant any small molecule chemical compound, antibody, nucleic acid molecule, or polypeptide, or fragments thereof. Exemplary agents include analgesics, angiogenic agents, antimicrobials, antibodies, antifungals, anti-inflammatories, anti-thrombotics, chemotherapeutics, growth factors, hormones, steroids.

By “ameliorate” is meant decrease, suppress, attenuate, diminish, arrest, or stabilize the development or progression of a disease.

By “alteration” is meant a change (increase or decrease) in the levels or activity of an analyte as detected by standard art known methods such as those described herein. As used herein, an alteration includes a 10% change in expression levels, preferably a 25% change, more preferably a 40% change, and most preferably a 50% or greater change in expression levels.

By “analog” is meant a molecule that is not identical, but has analogous functional or structural features.

By “antimicrobial” is meant an agent that inhibits or stabilizes the proliferation or survival of a microbe. In one embodiment, a bacteriostatic agent is an antimicrobial. In other embodiments, any agent that kills a microbe (e.g., bacterium, fungus, and virus) is an antimicrobial.

By “biodegradable” is meant susceptible to breakdown by biological activity. For example, biodegradable chitosan-PEGDMA compositions are susceptible to breakdown by enzymes present in vivo (e.g., lysozyme, N-acetyl-o-glucosaminidase and lipases). Degradation of a chitosan-PEGDMA composition of the invention need not be complete. A chitosan-PEGDMA composition of the invention may be degraded, for example, by the cleavage of one or more chemical bonds (e.g., glycosidic bonds).

By “clinician” is meant any healthcare provider. Exemplary clinicians include, but are not limited to, doctors, veterinarians, osteopaths, physician's assistants, emergency medical technicians, medics, nurse practitioners, and nurses.

The term “co-administration” or “combined administration” as used herein is defined to encompass the administration of the selected therapeutic agents to a single patient, and are intended to include treatment regimens in which the agents are not necessarily administered by the same route of administration or at the same time.

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

By “decreases” is meant a negative alteration of at least 10%, 25%, 50%, 75%, 100%, 200%, 300%, 400%, 500%, 1000%, or more.

“Detect” refers to identifying the presence, absence or amount of the analyte to be detected.

By “customize” is meant tailor to suit the needs of a particular subject.

By “degradation rate” is meant the time required to substantially degrade the composition. A composition is substantially degraded where at least about 75%, 85%, 90%, 95% or more has been degraded. Methods for measuring degradation of chitosan are known in the art and include measuring the amount of a microbead of the invention that remains following administration to a subject or following in vitro exposure to an enzyme having chitosan-degrading activity.

By “disease” is meant any condition or disorder that damages or interferes with the normal function of a cell, tissue, or organ. In one embodiment, the disease is a bacterial infection, fungal infection, or a combination there of present at a wound site. In another embodiment, the disease is a cancer.

By “effective amount” is meant the amount of an agent required to ameliorate the symptoms of a disease relative to an untreated patient. The effective amount of active agent(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “elution rate” is meant the time required for an agent to be substantially released from a composition. Elution can be measured by determining how much of an agent remains within the composition or by measuring how much of an agent has been released into the composition's surroundings. Elution may be partial (10%, 25%, 50%, 75%, 80%, 85%, 90%, 95% or more) or complete. In one preferred embodiment, the agent continues to be released at an effective level for at least about 3, 4, 5, 6, 7, 8, 9, or 10 days.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “infection” is meant the presence of one or more pathogens in a tissue or organ of a host. An infection includes the proliferation of a microbe (e.g., bacteria, viruses, fungi) within a tissue of a subject at a site of trauma.

By “increases” is meant a positive alteration of at least 10%, 25%, 50%, 75%, 100%, 200%, 300%, 400%, 500%, 1000%, or more.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “marker” is meant any protein or polynucleotide having an alteration in expression level or activity that is associated with a disease or disorder.

As used herein, “obtaining” as in “obtaining an agent” includes synthesizing, purchasing, or otherwise acquiring the agent.

By “point of treatment” is meant the site where healthcare is delivered. A “point of treatment” includes, but is not limited to, a surgical suite, physician's office, clinic, or hospital.

By “polymer” is meant a natural or synthetic organic molecule formed by combining smaller molecules in a regular pattern.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

By “profile” is meant a set of characteristics that define a composition or process. For example, a “biodegradation profile” refers to the biodegradation characteristics of a composition. In another example, an “elution profile” refers to elution characteristics of a composition.

By “reference” is meant a standard or control condition.

By “small molecule” is meant any chemical compound.

By “subject” is meant a mammal, including, but not limited to, a human or non-human mammal, such as a bovine, equine, canine, ovine, or feline.

By “trauma” is meant any injury that damages a tissue or organ of a subject. The injury need not be severe. Therefore, a trauma includes any injury that breaks the skin.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Any compounds, compositions, or methods provided herein can be combined with one or more of any of the other compositions and methods provided herein.

Unless specifically stated or obvious from context, as used herein, the term “or” is understood to be inclusive. Unless specifically stated or obvious from context, as used herein, the terms “a”, “an”, and “the” are understood to be singular or plural. Thus, for example, reference to “an amino acid substitution” includes reference to more than one amino acid substitution.

The term “including” is used herein to mean, and is used interchangeably with, the phrase “including but not limited to.”

As used herein, the terms “comprises,” “comprising,” “containing,” “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

Other features and advantages of the invention will be apparent from the following description of the desirable embodiments thereof, and from the claims.

### BRIEF DESCRIPTION OF THE DRAWINGS

FIG. 1 provides a schematic showing a conceptualized framework for the therapeutic agent delivery system responsive to external stimuli.

FIG. 2 provides a schematic depicting Brownian motion showing physical rotation of the particle along with the magnetization moment (top panel) and Neel relaxation where only magnetic moment flips (bottom panel). A and B represent two physical points on the particle, the white arrow signifies magnetization vector.

FIGS. 3A-3E show magnetic nanoparticle (MNP) data.

FIG. 3A provides a graph showing an X-Ray Diffraction (XRD) pattern of Fe3O4 magnetic nanoparticle.

FIG. 3B provides a graph showing a magnetization curve of Fe₃O⁴ magnetic nanoparticle.

FIG. 3C provides a Transmission Electron Microscopy (TEM) image of Magnetic Nanoparticle (MNP).

FIG. 3D provides a Scanning Electron Microscopy (SEM) image of chitosan microbeads with MNP.

FIG. 3E provides a SEM image of chitosan microbeads without MNP.

FIG. 3F provides a SEM image of stimulated and control MNP-loaded microbeads at various magnifications, showing that integrity of bead structure is not significantly changed after stimulation.

FIGS. 4A-4C show X-Ray Diffraction (XRD) plots for therapeutic agent delivery system components.

FIG. 4A provides a XRD plot of vancomycin.

FIG. 4B provides a XRD plot of chitosan with polyethylene dimethacrylate (PEGDMA) and vancomycin.

FIG. 4C provides a XRD plot chitosan with PEGDMA, vancomycin and MNP.

FIG. 5 provides a scatter plot where frequency and maximum field amplitude on the x axis and differential temperature rise (° C.) is represented on the y axis: Total temperature rise observed for 185 mg MNP in 2 mL PBS, stimulated for 10 minutes. Furthermore, the x-axis shows 5 different frequency/magnetic field intensity pairs recommended by the manufacturer.

FIGS. 6A-6B show data indicating the concentration of vancomycin over time with magnetic stimulation (experimental group) and without stimulation (control group) for varying durations. Data represented in FIGS. 6A and 6B is an average±standard deviation.

Asterisks (*) represent statistically significant differences between stimulated and control groups, p<0.05. In each pair of bars on the graph, the control bar is to the left and the experimental (i.e., Stim) is on the right. The first pair is labeled.

FIG. 6A provides a graph showing data short term from a elution study with stimulation given at 3, 5 and 7 hours.

FIG. 6B provides a graph showing data from a long term elution study with stimulus given on Day 12 and Day 15.

FIG. 7A provides a graph indicating the amount of vancomycin eluted from chitosan microbeads without magnetic nanoparticles. Assuming 5% confidence level, the difference in vancomycin elution was not significant between test and control groups. In each pair of bars on the graph, the control bar is to the left and the experimental (i.e., Stim) is on the right. The first pair is labeled.

FIG. 7B provides a scatter plot indicating the concentration of vancomycin released from chitosan microbeads with magnetic nanoparticles over time with and without magnetic stimulation on days 12 and 15. Lines represent effective minimum inhibitory values for vancomycin against S. aureus. Data points represent individual test values. Asterisks represent statistically significant differences between groups, p<0.05.

FIG. 8 provides photograph of the Magnetherm equipment for magnetic stimulation FIG. 9A provides a schematic diagram illustrating a timeline of hyperthermia experiments on samples with magnetic nanoparticles.

FIG. 9B provides a schematic diagram illustrating a timeline of hyperthermia experiments on samples without magnetic nanoparticles.

FIG. 9C provides a schematic diagram illustrating a timeline of hyperthermia experiments on samples without magnetic nanoparticles. Vertical arrows represent sampling instances for HPLC tests.

FIGS. 10A-10 show additional supporting data for the agent delivery system.

FIG. 10A provides a graph showing an exemplary controlled dose response curve and possible applications. FIG. 10B provides a schematic showing that the stimuli-responsiveness of chitosan composite is dependent on electrostatic interactions. Modalities for disrupting electrostatic interactions include, for example, changes in pH, temperature (e.g., application of heat), and enzymatic changes (e.g., degradation).

FIG. 10C provides a schematic showing the formation of microbeads comprising a cross-linked chitosan composite useful in drug delivery.

FIG. 10D provides a schematic showing that the PEG cross-linker may be more susceptible to heat or electromagnetic energy.

FIG. 10E provides a schematic showing the Michael addition reaction cross-linking provides controllable length and properties of cross-link.

FIG. 10F provides a schematic showing components of the therapeutic agent delivery system, including magnetic nanoparticle-loaded composite chitosan microbeads. In FIG. 10F, the agent is indicated by triangles, the MNP by circles, the chitosan by squiggly lines, and the cross link by straight lines.

FIG. 10G provides images showing the formation of porous beads with magnetic properties.

FIG. 10H provides a graph showing chitosan-magnetic nanoparticles beads increase in temperature after stimulation.

FIG. 10I provides images showing cytocompatible and biodegradable PEGDMA cross-linked beads.

FIG. 10J provides a graph showing data for vancomycin standards and elution samples that is useful in determining whether the drug is active after release and whether the therapeutic agent is tethered or free of conjugates. Experimental samples are identified using arrows in the graph on the far left.

FIG. 11A shows the experimental timeline for injection of chitosan-magnetic nanoparticle beads infused with rhodamine into mice.

FIG. 11B is a graph showing radiance in mice that received the injections of chitosan magnetic nanoparticle beads infused with rhodamine before and after a magnetic pulse in the control vs. test group. Both the control and test groups was injected with chitosan-magnetic nanoparticle beads infused with rhodamine, but only the test group received the magnetic pulse. “Before” denotes prior to the magnetic pulse and “After” denotes after the magnetic pulse. A significant release of rhodamine occurred following the magnetic pulse on days 2, 3 and 4.

FIG. 11C shows radiance in mice that received the injections of chitosan magnetic nanoparticle beads infused with rhodamine before and after a magnetic pulse. A significant release of rhodamine is observed following the magnetic pulse in the test mice before and after on day one stimulation.

### DETAILED DESCRIPTION OF THE INVENTION

As described below, the present invention provides chitosan microbeads comprising a therapeutic agent and methods of using the microbeads for drug delivery in response to an external stimulus.

The invention is based, at least in part, on the discovery that chitosan microbeads comprising magnetic nanoparticles release a therapeutic agent in response to magnetic stimulation. Local antibiotic delivery can overcome some of the shortcomings of systemic therapy, such as low local concentrations and delivery to avascular sites. A localized therapeutic agent delivery system, ideally, could also use external stimuli to modify the normal drug release profile from the therapeutic agent delivery system to provide efficacious drug administration flexibility to healthcare providers. As reported in more detailed below, to achieve this motive, chitosan microbeads embedded with magnetic nanoparticles were loaded with vancomycin antibiotic and stimulated by a high frequency alternating magnetic field. Repeated stimulation sessions, separated by several hours, were carried out. The chromatographic analysis of the supernatant from these stimulated samples showed more than ˜200% higher release of vancomycin from the therapeutic agent delivery system after the stimulation periods compared to control samples. A long term elution study was carried out where the therapeutic agent delivery system was allowed to elute drug over a period of 11 days and stimulated on day 12 and day 15, when vancomycin level dropped below therapeutic levels. The stimuli were effective in boosting elution of test groups above MIC, as compared to control groups which had almost nil elution in the stimulation span. Interestingly, the drug release between test and control groups seemed to be similar in the intervals without excitation. The results indicate a stimuli-responsive therapeutic agent delivery system controllable by magnetic excitation.

Accordingly, the invention provides microbeads (e.g., chitosan-PEGDMA) compositions comprising a therapeutic agent or combination of therapeutic agents (e.g., analgesics, angiogenic agents, antimicrobials, antibodies, antifungals, anti-inflammatories, anti-thrombotics, chemotherapeutics, growth factors, hormones, steroids) for the treatment or prevention of an infection, disease, or medical condition.

The present invention is capable of releasing a therapeutic agent in response to an external stimuli. The composition of the present invention is nontoxic and biodegradable, and well suited for use with implantable therapeutic agent delivery applications.

In one embodiment, chitosan cross-linking is accomplished through the use of a Michael addition reaction, which can provide control over the length and properties of the cross-link formed. The cross-linking reaction does not require the use of toxic initiators to react the amino groups of chitosan and PEGDMA derivatives. Furthermore, the length of the linker can affect the release rate and/or pattern of release of therapeutic agents. In one embodiment, the linker regions can incorporate functionality for near infrared or enzymatic cleavage of the linkage. Suitable cross-linking polymers include any polymer with (meth)acrylated ends, difunctional or multifunctional end groups.

Existing technologies for drug delivery typically rely on direct pharmaceutical injection or oral administration. In some cases, the therapeutic agent is encapsulated in a therapeutic agent delivery system that is intended to release the payload at the location of interest. Conventionally, drug elution occurs in response to in vivo physiological conditions, while other therapeutic agent delivery systems employ an extrinsic stimulus to cause a single burst discharge of the agent. In contrast, an advantage of the present invention lies in the fact that it releases agent over an extended period of time in response to external stimuli.

Experiments were conducted to determine the suitability of the present invention for therapeutic use. Examination of the morphology of stimulated microbeads subsequent to stimulation showed that the bead remained intact and structurally sound after multiple magnetic or electric field pulses. In the extended duration tests presented herein, stimulation triggered detectable release of a therapeutic agent (e.g., antibiotic-vancomycin). This treatment approach provides clinicians with unprecedented control over the agent release, such that the agent release can be turned “on” or “off” by a non-invasive stimulus. The present invention provides for the delivery of multiple doses of an agent at discrete intervals, without the discomfort of invasive administration. Moreover, because chitosan microbeads contain magnetic nanoparticles they can be visualized using, for example, an MM. This provides real-time location status via MM.

### Therapeutic Agent Delivery Systems

In the past few decades, pharmaceutical research has progressed significantly in various smart therapeutic agent delivery systems that are aimed at controlling dosage and localization of therapeutic agents. Various therapeutic agent delivery systems are discussed by Mohapatra et al. [1] (Stealth Engineering for in vivo Drug Delivery Systems”. Critical Reviews in Biomedical Engineering, vol. 43, pp. 347-69, 2016.), which is incorporated by reference in its entirety.

Traditional systemic delivery of antibiotics is not always effective in achieving minimum inhibitory concentration (MIC) required for sustained treatment at the target site of injured tissue, thereby requiring stronger or repetitive dosages for efficacy in preventing infection [2]. The reason for insufficient concentration is usually due to avascular nature of injured tissue, dissipation of drug into non-targeted tissues, dilution due to vascular circulation, or opsonisation by Mononuclear Phagocytic System (MPS). There have been numerous approaches to enhance pharmacokinetic efficiency and longevity of pharmaceutical products in vivo. A popular method is to use a biocompatible, biodegradable therapeutic agent delivery system which allows longer bioavailability and localization of drug at potent concentrations at the site of interest. Several of these therapeutic agent delivery systems have been approved by FDA for clinical use.

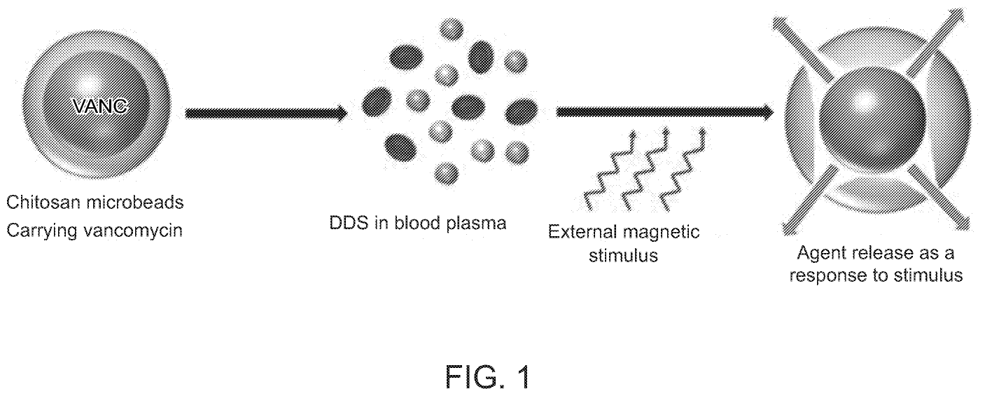

Although these therapeutic agent delivery systems prolong the lifetime and efficacy of drug compared to naked drug, these therapeutic agent delivery systems characteristically have a continuous first-order elution profile until the payload is exhausted [3]. Several modifications have been progressively proposed to make such therapeutic agent delivery systems responsive to a variety of stimuli, which would enable on-demand dosage optimization to actual therapeutic requirement [4-6]. Also, a drug boost might be required later than when the therapeutic agent delivery system is implanted. The data presented herein indicates applying a high-frequency alternating magnetic field as an exogenous stimulus to cause drug release in a therapeutic agent delivery system through magnetic hyperthermia (FIG. 1).

The therapeutic agent delivery system of the present invention is in the form of microbeads structured from chitosan, cross-linked with polyethylene dimethacrylate (PEGDMA) and embedded with magnetic nanoparticles (MNPs). In the experiments presented herein, vancomycin, an antibiotic, was loaded into them to study drug release profiles. Data supporting a multiple pulsatile drug release phases controllable by alternating external magnetic fields was obtained, establishing a proof of principle. A long term study where stimulation was applied after several days and succeeded in increasing elution significantly. This can potentially have the benefit of using a non-invasive form of stimulus to maintain the antibiotic concentration in a way that is most efficient to fight infection.

### Magnetic Hyperthermia Theory

Hyperthermia, in generic terms, is described as an elevation in temperature. In magnetic structures, hyperthermia by high frequency magnetic fields can be attributed to power losses caused by eddy currents, hysteresis, Brownian or Néel relaxation [7-11].

Eddy current losses are significant only in larger bulk materials, therefore can be ignored for MNP [7-11]. For single-domain particles with size below the critical volume Vc, hysteresis losses decrease abruptly, being suppressed by relaxation effects.

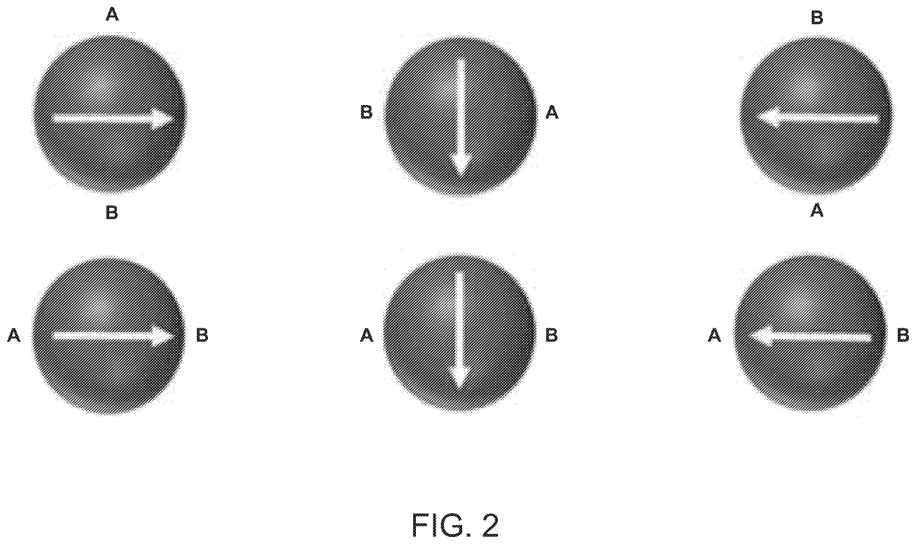

In Brownian relaxation, the particle rotates while its magnetization vector stays fixed relative to crystalline axes, generating frictional losses (FIG. 2) [10-12]. The Brownian relaxation time is given by τ_{B}=4πηrₖ [symbol] kt. Wherein the terms are defined by: rₖ: hydrodynamic radius, η: viscosity of suspension medium, kT: Thermal energy.

In Neel relaxation, the particle remains physically fixed while its magnetic moment direction reorients against an anisotropic energy barrier, dissipating thermal energy (FIG. 2) [10-12]. The Néel relaxation time is mathematically defined as:

$$\text{?} = \text{?} exp ( \frac{\text{?}}{\text{?}} ) . \\ \text{?} \text{indicates text missing or illegible when filed}$$

Wherein the terms are defined by: K: anisotropy constant, V: Volume of particle, τ₀: constant, ˜10⁻⁹, KV: height of energy barrier.

Usually both phenomena are active and effective relaxation time is estimated as given in the following equation: τ_{eff}=τ_{N}τ_{B}/(τ_{N}+τ_{B}). The phenomena with the smaller time constant dominates the relaxation mode.

Rosenweig formulated an equation of power loss caused due to these relaxation as shown below [12]: F=πμ₀X₀H₀²f*2πfτ_{eff}/(1+(2πfτ_{eff})². Wherein the terms are defined by: X₀: magnetic susceptibility, f: frequency of magnetic field, H₀: amplitude of magnetic field.

Hyperthermia for drug release was first adopted and successfully demonstrated in 1987 when diabetic rats were implanted with polymeric matrices loaded with insulin and embedded magnets. Although passive elution of insulin induced lowering of glucose levels in all mice, a stimulus of magnetic field was observed to cause a further drop in glucose level by 30% [13]. Hyperthermia has since been extensively explored for smart therapeutic applications like cancer therapy and therapeutic agent delivery, leading to successful clinical trials on human subjects with brain/prostate cancer [14]. Using magnetic targeting and intracarotid delivery of MNP, a 30-fold increase in particle entrapment by brain tumor was recorded compared to traditional intravenous administration [15]. Also, since MNP, like magnetite, have been proven to be efficient MM contrast agents [16-20], they facilitate drug targeting, localization and confinement [15,20-21]. Finotelli et al. loaded insulin in alginate/chitosan microbeads with magnetite nanoparticles and showed insulin release tripled with respect to control when a magnetic field of 1800 G, 33 Hz was applied to the test groups [22].

Hu et al. constructed Fe3O4/poly (allylamine) polyelectrolyte microcapsules, loaded with doxorubicin hydrochloride. On application of a high frequency magnetic field, micro-cavities appeared on the therapeutic agent delivery system surface and exacerbated into major ruptures with time, eluting drug in significant amounts [23]. Koppolu et al. designed MNP cores with outer multilayered shells of the temperature-responsive polymer poly(N-isopropylacrylamaide) (PNIPAAm) and poly(D,L-lactideco-glycolide) (PLGA) as carriers of both curcumin and bovine serum albumin (BSA); while curcumin showed a sustained release profile over 13 d, BSA could be burst-released from PNIPAAm layer by elevating temperature [24]. Katagiri and his group designed polyelectrolyte hollow multilayered shells containing dye, coated with Fe3O4 MNP and an amphiphilic bilayer. They magnetically irradiated at 236 Oersted, 360 kHz for 60 min and measured dye release, which was associated with a heat-induced change in phase of amphiphilic membrane, rather than any structural fissure [25].

### Chitosan-PEGDMA Compositions

Chitosan is a naturally occurring linear polysaccharide composed of randomly distributed B-(1-4)-2-amino-2-D-glucosamine (deacetylated) and B-(1-4)-2-acetamido-2-D-glucoseamine (acetylated) units and is cationic by nature. Chitosan is derived from chitin, a naturally occurring polymer. Chitin is a white, hard, inelastic, nitrogenous polysaccharide isolated from fungi, mollusks, or from the exoskeletons of arthropods (e.g., crustaceans, insects). The major procedure for obtaining chitosan is the alkaline deacetylation of chitin with strong alkaline solution. Generally, the raw material is crushed, washed with water or detergent, and ground into small pieces. After grinding, the raw material is treated with alkali and acid to isolate the polymer from the raw crushed material. The polymer is then deacetylated by treatment with alkali. Chitin and chitosan differ in their degrees of deacetylation (DDA). Chitin has a degree of deacetylation of 0% while pure chitosan has a degree of deacetylation of 100%. Typically, when the degree of deacetylation is greater than about 50% the polymer is referred to as chitosan.

Chitosan is a cationic weak base that is substantially insoluble in water and organic solvents. Typically, chitosan is fairly soluble in dilute acid solutions, such as acetic, citric, oxalic, proprionic, ascorbic, hydrochloric, formic, and lactic acids, as well as other organic and inorganic acids. Chitosan's charge gives it bioadhesive properties that allow it to bind to negatively charged surfaces, such as biological tissues present at a site of trauma or negatively charged implanted devices.

Chitosan's degree of deacetylation affects it resorption. As the degree of deacetylation increases, chitosan becomes increasingly resistant to degradation. Chitosan-PEGDMA compositions having a degree of deacetylation that is higher than 95% degrade slowly over weeks or months. In the body chitosan is degraded by lysozyme, N-acetyl-o-glucosaminidase, and lipases. Lysozyme degrades chitosan by cleaving the glycosidic bonds between the repeating chitosan units. The byproducts of chitosan degradation are saccharides and glucosamines that are gradually absorbed by the human body. Therefore, when chitosan is used for the local delivery of therapeutic or prophylactic agents, no secondary removal operation is required.

Chitosan has been widely researched as a component of therapeutic agent delivery systems because of positive characteristics such as biodegradability, non-cytotoxicity, intracellular permeability, biocompatibility, and its ability to entrap drugs [26-29]. Although there are different modified combinations of chitosan with MNP that have been investigated for use in general localized hyperthermia caused by magnetic fields [30-32], or as MRI contrast agents [33-36], a formulation devised for controllable therapeutic agent delivery by magnetic field has not been documented yet.

As reported herein, chitosan-polyethylene dimethacrylate (PEGDMA) compositions were prepared that can be loaded with therapeutic agents, including antibiotic agents such as vancomycin. The weight percentage of total polymer (e.g., comprising chitosan and PEGDMA) is at least about 1-2%. In particular embodiments, the weight percentage of total polymer is 1%. The ratio of chitosan:PEGDMA may range from about 1:1 to 4:1. In particular embodiments, a chitosan:PEG ratio of 1:1 is used. In some embodiments, the molecular weight of PEG is about 6,000-10,000 g/mol. In various embodiments, the PEG used is 6,000 or 8,000 g/mol. In some embodiments, the molecular weight of PEG is about 200-10,000 g/mol. In various embodiments, the PEG used is 750 or 1,000 g/mol. The chitosan used has a DDA between about 61% to 85%. In certain embodiments, the chitosan used has a DDA of about 61% or 71%. In other embodiments, the final sponge formulations used chitosan with 82.46±1.679% DDA.

The chitosan-PEGDMA compositions of the invention (e.g., solids, hydrogels, and composites) can be loaded with one or more biologically active agents.

In one embodiment, the degree of deacetylation is adjusted to provide chitosan-PEGDMA compositions that degrade in as little as about twenty-four, thirty-six, forty-eight, or seventy two hours or that are maintained for a longer period of time (e.g., 4, 5, 6, 7, 8, 9, 10 days). In other embodiments, chitosan-PEGDMA compositions of the invention are maintained in the body for at least about two-six weeks or more (e.g., 2, 3, 4, 5, 6 weeks, two, three or four months). In still other embodiments, chitosan-PEGDMA compositions of the invention enhance blood clotting in a wound or other site of trauma (hemostasis).

In other embodiments, the chitosan-PEGDMA compositions are loaded with therapeutic or prophylactic agents that are clinician selected and that are delivered over at least about 1, 2, 3, 4, 5, 6, 7, 8, 9, or 10 days or for longer periods.

In other embodiments, the chitosan-PEGDMA compositions are loaded with therapeutic or prophylactic agents that are clinician selected and that are delivered over at least about 1, 2, 3, 4, 5, 6, 7, 8, 9, or 10 days or for longer periods.

As described herein, the experimental results demonstrated that blending chitosan microbeads with polyethylene dimethacrylate (PEGDMA) and Magnetic Nanoparticles (MNPs) in a therapeutic composition significantly affected compositions material properties and vancomycin elution properties.

Research has been minimal on local therapeutic agent delivery systems, and many of the local delivery systems that exist release too little therapeutic agent, the system does not adequately provide for repeated release of therapeutic agent, or the system is not designed to degrade.

The present results indicate that blending cross linked chitosan and PEGDMA with magnetic nanoparticles creates biocompatible and degradable compositions.

### Crosslinking Polymers

The invention provides chitosan microbeads that are cross-linked with a polymer, such as PEG or PEGDMA, and embedded with magnetic nanoparticles. In particular embodiments, chitosan microbeads are cross-linked with virtually any polymer known in the art. Polymers can include polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, and cellulose sulphate sodium salt. In yet other embodiments the cross linking polymer contains any one or more of the following polymers: poly(methyl methacrylate), poly(ethylmethacrylate), poly(butylmethacrylate), poly(isobutylmethacrylate), poly(hexlmethacrylate), poly(isodecylmethacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene poly(ethylene glycol), poly(ethylene oxide), and poly(ethylene terephthalate). In yet other embodiments, the cross linking polymer contains any one or more of the following polymers: poly(vinyl alcohols), poly(vinyl acetate, poly vinyl chloride polystyrene, polyvinylpryrrolidone, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutylmethacrylate), poly(hexlmethacrylate), poly(isodecl methacrylate), poly(lauryl methacrylate), poly(phenyl methacrylate), poly(methyl acrylate), poly(isopropyl acrylate), poly(isobutyl acrylate), and poly(octadecl acrylate). In one embodiment, the polymer is a polyethylene dimethacrylate (PEGDMA) polymer.

### Magnetic Nanoparticles (MNPs)

Magnetic Nanoparticles (MNPs) are incorporated into the therapeutic agent delivery system of the present invention. The magnetic nanoparticles can be responsive to the application of magnetic, thermal, chemical, enzymatic or other stimuli. Nanoparticles, related therapeutic compositions, and methods of preparing magnetic nanoparticles are described by U.S. Pat. Nos. 9,330,821, 8,746,999, 8,563,020, 8,535,640, 7,988,949, U.S. Patent Application No. 20130209537 and International Patent Application Nos. WO 2012109121, WO 2012003432, WO 2010063998, WO 2009108407, WO 2008075222, which are incorporated herein by reference in their entirety.

The mass of the magnetic nanoparticle can be about 1-150 KD (e.g., any integer between about 1 and 150, where the bottom of the range is any integer between about 1 and 149, and the top of the range is any integer between about 2 and 150). In one embodiment, the mass of the nanoparticle is about 30-60 KD (e.g., about 30, 35, 40, 45, 50, 55, or 60). The size of the nanoparticle is about 1-500 nm (e.g., about 100-400 nm, 200-300 nm, or 10-100), where the bottom of the range is any integer between about 1-499 and the top of the range is any integer between about 2 and 500.

### Antimicrobial Agents

The invention provides chitosan microbeads comprising a therapeutic agent. In particular, chitosan microbeads comprise an antimicrobial useful for treating an infection. Staphylococcus aureus, Staphylococcus epidermidis, Pseudomonas aeruginosa and Candida albicans are pathogens that are commonly present at musculoskeletal wound sites. S. aureus is one cause of osteomyelitis and nongonococcal bacterial arthritis, and is often associated with prosthetic joint infection. The invention provides chitosan-PEGDMA compositions useful in treating or preventing infection in a wound, complex wound, open fraction, or other site of trauma. Any antimicrobial agent known in the art can be used in the chitosan-PEGDMA compositions of the invention at concentrations generally used for such agents.

Antimicrobial agents useful in chitosan microbead (e.g., chitosan-PEGDMA) compositions of the invention include but are not limited to antibacterials, antifungals, and antivirals. An antimicrobial agent as used herein is an agent which reduces or stabilizes the survival, growth, or proliferation of a pathogen. Antimicrobial agents include but are not limited to Aztreonam; Chlorhexidine Gluconate; Imidurea; Lycetamine; Nibroxane; Pirazmonam Sodium; Propionic Acid; Pyrithione Sodium; Sanguinarium Chloride; Tigemonam Dicholine; Acedapsone; Acetosulfone Sodium; Alamecin; Alexidine; Amdinocillin; Amdinocillin Pivoxil; Amicycline; Amifloxacin; Amifloxacin Mesylate; Amikacin; Amikacin Sulfate; Aminosalicylic acid; Aminosalicylate sodium; Amoxicillin; Amphomycin; Ampicillin; Ampicillin Sodium; Apalcillin Sodium; Apramycin; Aspartocin; Astromicin Sulfate; Avilamycin; Avoparcin; Azithromycin; Azlocillin; Azlocillin Sodium; Bacampicillin Hydrochloride; Bacitracin; Bacitracin Methylene Disalicylate; Bacitracin Zinc; Bambermycins; Benzoylpas Calcium; Berythromycin; Betamicin Sulfate; Biapenem; Biniramycin; Biphenamine Hydrochloride; Bispyrithione Magsulfex; Butikacin; Butirosin Sulfate; Capreomycin Sulfate; Carbadox; Carbenicillin Disodium; Carbenicillin Indanyl Sodium; Carbenicillin Phenyl Sodium; Carbenicillin Potassium; Carumonam Sodium; Cefaclor; Cefadroxil; Cefamandole; Cefamandole Nafate; Cefamandole Sodium; Cefaparole; Cefatrizine; Cefazaflur Sodium; Cefazolin; Cefazolin Sodium; Cefbuperazone; Cefdinir; Cefepime; Cefepime Hydrochloride; Cefetecol; Cefixime; Cefinenoxime Hydrochloride; Cefmetazole; Cefmetazole Sodium; Cefonicid Monosodium; Cefonicid Sodium; Cefoperazone Sodium; Ceforanide; Cefotaxime Sodium; Cefotetan; Cefotetan Disodium; Cefotiam Hydrochloride; Cefoxitin; Cefoxitin Sodium; Cefpimizole; Cefpimizole Sodium; Cefpiramide; Cefpiramide Sodium; Cefpirome Sulfate; Cefpodoxime Proxetil; Cefprozil; Cefroxadine; Cefsulodin Sodium; Ceftazidime; Ceftibuten; Ceftizoxime Sodium; Ceftriaxone Sodium; Cefuroxime; Cefuroxime Axetil; Cefuroxime Pivoxetil; Cefuroxime Sodium; Cephacetrile Sodium; Cephalexin; Cephalexin Hydrochloride, Cephaloglycin; Cephaloridine; Cephalothin Sodium; Cephapirin Sodium; Cephradine; Cetocycline Hydrochloride; Cetophenicol; Chloramphenicol; Chloramphenicol Palmitate; Chloramphenicol Pantothenate Complex; Chloramphenicol Sodium Succinate; Chlorhexidine Phosphanilate; Chloroxylenol; Chlortetracycline Bisulfate; Chlortetracycline Hydrochloride; Cinoxacin; Ciprofloxacin; Ciprofloxacin Hydrochloride; Cirolemycin; Clarithromycin; Clinafloxacin Hydrochloride; Clindamycin; Clindamycin Hydrochloride; Clindamycin Palmitate Hydrochloride; Clindamycin Phosphate; Clofazimine; Cloxacillin Benzathine; Cloxacillin Sodium; Cloxyquin; Colistimethate Sodium; Colistin Sulfate; Coumermycin; Coumermycin Sodium; Cyclacillin; Cycloserine; Dalfopristin; Dapsone; Daptomycin; Demeclocycline; Demeclocycline Hydrochloride; Demecycline; Denofungin; Diaveridine; Dicloxacillin; Dicloxacillin Sodium; Dihydrostreptomycin Sulfate; Dipyrithione; Dirithromycin; Doxycycline; Doxycycline Calcium; Doxycycline Fosfatex; Doxycycline Hyclate; Droxacin Sodium; Enoxacin; Epicillin; Epitetracycline Hydrochloride; Erythromycin; Erythromycin Acistrate; Erythromycin Estolate; Erythromycin Ethylsuccinate; Erythromycin Gluceptate; Erythromycin Lactobionate; Erythromycin Propionate; Erythromycin Stearate; Ethambutol Hydrochloride; Ethionamide; Fleroxacin; Floxacillin; Fludalanine; Flumequine; Fosfomycin; Fosfomycin Tromethamine; Fumoxicillin; Furazolium Chloride; Furazolium Tartrate; Fusidate Sodium; Fusidic Acid; Gentamicin Sulfate; Gloximonam; Gramicidin; Haloprogin; Hetacillin; Hetacillin Potassium; Hexedine; Ibafloxacin; Imipenem; Isoconazole; Isepamicin; Isoniazid; Josamycin; Kanamycin Sulfate; Kitasamycin; Levofuraltadone; Levopropylcillin Potassium; Lexithromycin; Lincomycin; Lincomycin Hydrochloride; Lomefloxacin; Lomefloxacin Hydrochloride; Lomefloxacin Mesylate; Loracarbef; Mafenide; Meclocycline; Meclocycline Sulfosalicylate; Megalomicin Potassium Phosphate; Mequidox; Meropenem; Methacycline; Methacycline Hydrochloride; Methenamine; Methenamine Hippurate; Methenamine Mandelate; Methicillin Sodium; Metioprim; Metronidazole Hydrochloride; Metronidazole Phosphate; Mezlocillin; Mezlocillin Sodium; Minocycline; Minocycline Hydrochloride; Mirincamycin lydrochloride; Monensin; Monensin Sodium; Nafcillin Sodium; Nalidixate Sodium; Nalidixic Acid; Natamycin; Nebramycin; Neomycin Palmitate; Neomycin Sulfate; Neomycin Undecylenate; Netilmicin Sulfate; Neutramycin; Nifuradene; Nifuraldezone; Nifuratel; Nifuratrone; Nifurdazil; Nifurimide; Nifurpirinol; Nifurquinazol; Nifurthiazole; Nitrocycline; Nitrofurantoin; Nitromide; Norfloxacin; Novobiocin Sodium; Ofloxacin; Ormetoprim; Oxacillin Sodium; Oximonam; Oximonam Sodium; Oxolinic Acid; Oxytetracycline; Oxytetracycline Calcium; Oxytetracycline Hydrochloride; Paldimycin; Parachlorophenol; Paulomycin; Pefloxacin; Pefloxacin Mesylate; Penamecillin; Penicillin G Benzathine; Penicillin G Potassium; Penicillin G Procaine; Penicillin G Sodium; Penicillin V; Penicillin V Benzathine; Penicillin V Hydrabamine; Penicillin Y Potassium; Pentizidone Sodium; Phenyl Aminosalicylate; Piperacillin Sodium; Pirbenicillin Sodium; Piridicillin Sodium; Pirlimycin Hydrochloride; Pivampicillin Hydrochloride; Pivampicillin Pamoate; Pivampicillin Probenate; Polymyxin B Sulfate; Porfiromycin; Propikacin; Pyrazinamide; Pyrithione Zinc; Quindecamine Acetate; Quinupristin; Racephenicol; Ramoplanin; Ranimycin; Relomycin; Repromicin; Rifabutin; Rifametane; Rifamexil; Rifamide; Rifampin; Rifapentine; Rifaximin; Rolitetracycline; Rolitetracycline Nitrate; Rosaramicin; Rosaramicin Butyrate; Rosaramicin Propionate; Rosaramicin Sodium Phosphate; Rosaramicin Stearate; Rosoxacil; Roxarsone; Roxithromycin; Sancycline; Sanfetrinem Sodium; Sarmoxicillin; Sarpicillin; Scopafungin; Sisomicin; Sisomicin Sulfate; Sparfloxacin; Spectinomycin Hydrochloride; Spiramycin; Stallimycin Hydrochloride; Steffimycin; Streptomycin Sulfate; Streptonicozid; Sulfabenz: Sulfabenzamide; Sulfacetamide; Sulfacetamide Sodium; Sulfacytine; Sulfadiazine; Sulfadiazine Sodium; Sulfadoxine; Sulfalene; Sulfamerazine; Sulfameter; Sulfamethazine; Sulfamethizole; Sulfamethoxazole; Sulfamonomethoxine; Sulfamoxole; Sulfanilate Zinc; Sulfanitran; Sulfasalazine; Sulfasomizole; Sulfathiazole; Sulfazamet; Sulfisoxazole; Sulfisoxazole Acetyl; Sulfisoxazole Diolamine; Sulfomyxin; Sulopenem; Sultamicillin; Suncillin Sodium; Talampicillin Hydrochloride; Teicoplanin; Temafloxacin Hydrochloride; Temocillin; Tetracycline; Tetracycline Hydrochloride; Tetracycline Phosphate Complex; Tetroxoprim; Thiamphenicol; Thiphencillin Potassium; Ticarcillin Cresyl Sodium: Ticarcillin Disodium; Ticarcillin Monosodium; Ticlatone; Tiodonium Chloride; Tobramycin; Tobramycin Sulfate; Tosufloxacin; Trimethoprim; Trimethoprim Sulfate; Trisulfapyrimidines; Troleandomycin; Trospectomycin Sulfate; Tyrothricin; Vancomycin; Vancomycin Hydrochloride; Virginiamycin; Zorbamycin; Difloxacin Hydrochloride; Lauryl Isoquinolinium Bromide; Moxalactam Disodium; Ornidazole; Pentisomicin; and Sarafloxacin Hydrochloride.

In one embodiment, vancomycin was chosen as the drug of interest. It is a glycopeptide antibiotic that is potent against gram-positive bacteria and is used to treat streptococcal and staphylococcal strains [37].

It is known that antibiotics need to maintain a minimum inhibitory concentration to effectively eliminate infection. In work described herein, chitosan was used as the substrate for vancomycin. This allows a controllable release of the drug when a higher concentration is required at later time points. It also aids in localizing the drug in the target area. In a study, the half-life of intra-articular administration of vancomycin was measured to be 3 h [38]. The therapeutic levels in joint and serum were maintained for 24 h. Several attempts have been reported to extend the lifetime of vancomycin in vivo, including encapsulation in chitosan. Matripragada and Jayasuriya used chitosan microparticles as a substrate to slowly release vancomycin, cefazolin and bone morphogenetic proteins over two weeks [39]. Cerchiara et al designed a chitosan/carboxymethyl-cellulose complex microparticle for delivering vancomycin to colon. The therapeutic agent delivery system not only prevented premature degradation of the drug but also prolonged its bioactive duration against S. aureus [40]. Chitosan based vancomycin liposomes were also observed to have longer retention spans and better antibiotic efficacy compared to vancomycin injection [41].

### Analgesics

In other embodiments, a chitosan microbead (e.g., chitosan-PEGDMA) composition of the invention can be used for the delivery of one or more agents that ameliorate pain, such agents include but are not limited to opioid analgesics (e.g. morphine, hydromorphone, oxymorphone, levorphanol, levallorphan, methadone, meperidine, fentanyl, codeine, dihydrocodeine, oxycodone, hydrocodone, propoxyphene, nalmefene, nalorphine, naloxone, naltrexone, buprenorphine, butorphanol, nalbuphine or pentazocine; a nonsteroidal antiinflammatory drug (NSAID) (e.g., aspirin, diclofenac, diflusinal, etodolac, fenbufen, fenoprofen, flufenisal, flurbiprofen, ibuprofen, indomethacin, ketoprofen, ketorolac, meclofenamic acid, mefenamic acid, nabumetone, naproxen, oxaprozin, phenylbutazone, piroxicam, sulindac, tolmetin or zomepirac, or a pharmaceutically acceptable salt thereof; a barbiturate sedative, e.g. amobarbital, aprobarbital, butabarbital, butabital, mephobarbital, metharbital, methohexital, pentobarbital, phenobartital, secobarbital, talbutal, theamylal or thiopental or a pharmaceutically acceptable salt thereof; a COX-2 inhibitor (e.g. celecoxib, rofecoxib or valdecoxib).

### Angiogenic Agents

Angiogenic agents can include but are not limited to VEGF, PDGF, bFGF, TGF-β, placental growth factor (PIGF/PGF), angiopoietin (Ang)-2, angiogenin ephrin, and plasminogen activators.

### Chemotherapeutics

In particular embodiments, chitosan microbead compositions comprise chemotherapeutic agents including, but not limited to, alemtuzumab, altretamine, aminoglutethimide, amsacrine, anastrozole, azacitidine, bleomycin, bicalutamide, busulfan, capecitabine, carboplatin, carmustine, celecoxib, chlorambucil, 2-chlorodeoxyadenosine, cisplatin, colchicine, cyclophosphamide, cytarabine, cytoxan, dacarbazine, dactinomycin, daunorubicin, docetaxel, doxorubicin, epirubicin, estramustine phosphate, etodolac, etoposide, exemestane, floxuridine, fludarabine, 5-fluorouracil, flutamide, formestane, gemcitabine, gentuzumab, goserelin, hexamethylmelamine, hydroxyurea, hypericin, ifosfamide, imatinib, interferon, irinotecan, letrozole, leuporelin, lomustine, mechlorethamine, melphalen, mercaptopurine, 6-mercaptopurine, methotrexate, mitomycin, mitotane, mitoxantrone, nilutamide, nocodazole, paclitaxel, pentostatin, procarbazine, raltitrexed, rituximab, rofecoxib, streptozocin, tamoxifen, temozolomide, teniposide, 6-thioguanine, topotecan, toremofine, trastuzumab, vinblastine, vincristine, vindesine, and vinorelbine.

### Anti-Thrombotic Agents

In particular embodiments, chitosan microbead (e.g., chitosan-PEGDMA) compositions of the invention are also useful for inhibiting, reducing or ameliorating clot formation. In one embodiment, a chitosan-PEGDMA composition contains one or more anti-thrombotics (e.g., thrombin, fibrinogen, coumadin, and heparin).

### Anti-Inflammatory Agents

In other embodiments, a chitosan microbead (e.g., chitosan-PEGDMA) composition is used to deliver an antiinflammatory agent. Such anti-inflammatory agents include, but are not limited to, Alclofenac; Alclometasone Dipropionate; Algestone Acetonide; Alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lornoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Morniflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin Sodium; Triclonide; Triflumidate; Zidometacin; and Zomepirac Sodium.

### Growth Factors

In other embodiments, a chitosan microbead comprises a growth factor. Growth factors are typically polypeptides or fragments thereof that support the survival, growth, or differentiation of a cell. Such agents may be used to promote wound healing. A chitosan-PEGDMA composition described herein can be used to deliver virtually any growth factor known in the art. Such growth factors include but are not limited to angiopoietin, acidic fibroblast growth factors (aFGF) (GenBank Accession No. NP_149127) and basic FGF (GenBank Accession No. AAA52448), bone morphogenic protein (BMP)(GenBank Accession No. BAD92827), vascular endothelial growth factor (YEGF) (GenBank Accession No. AAA35789 or NP_001020539), epidermal growth factor (EGF) (GenBank Accession No. NP_001954), transforming growth factor a (TGF-a) (GenBank Accession No. NP_003227) and transforming growth factor (3 (TFG-(3) (GenBank Accession No. 1109243A), platelet-derived endothelial cell growth factor (PD-ECGF)(GenBank Accession No. NP_001944), platelet-derived growth factor (PDGF)(GenBank Accession No. 1109245A), tumor necrosis factor a (TNF-α)(GenBank Accession No. CAA26669), hepatocyte growth factor (HGF)(GenBank Accession No. BAA14348), insulin like growth factor (IGF)(GenBank Accession No. P08833), erythropoietin (GenBank Accession No. P01588), colony stimulating factor (CSF), macrophage-CSF (M-CSF)(GenBank Accession No. AAB59527), granulocyte/macrophage CSF (GM-CSF) (GenBank Accession No. NP_000749) and nitric oxide synthase (NOS)(GenBank Accession No. AAA36365). In one preferred embodiment, the growth factor is BMP.

### Hormones and Steroids

In other embodiments, a chitosan microbead comprises a hormone (e.g., insulin). Suitable hormones and steroids can include, for example, aldosterone, androstenedione, calcidiol, calcitriol, estradiol or estrogens, cortisol, dehydroepiandrosterone, dihydrotestosterone, testosterone, progesterone. Suitable hormones can include, for example, amylin, anti-Müllerian hormone, adiponectin, adrenocorticotropic hormone (or corticotropin), angiotensinogen, angiotensin, antidiuretic hormone (e.g., vasopressin, arginine vasopressin), atrial-natriuretic peptide (e.g., atriopeptin), brain natriuretic peptide, calcitonin, cholecystokinin, corticotropin-releasing hormone, cortistatin, encephalin, endothelin, erythropoietin, follicle-stimulating hormone, galanin, gastric inhibitory polypeptide, gastrin, ghrelin, glucagon, glucagon-like peptide-1, gonadotropin-releasing hormone, growth hormone-releasing hormone, growth hormone, hepcidin, human chorionic gonadotropin, human placental lactogen, inhibin, insulin, insulin-like growth factor (or somatomedin), leptin, lipotropin, luteinizing hormone, melanocyte stimulating hormone, motilin, orexin, oxytocin, pancreatic polypeptide, parathyroid hormone, pituitary adenylate cyclase-activating peptide, prolactin, prolactin releasing hormone, relaxin, renin, secretin, somatostatin, thrombopoietin, thyroid-stimulating hormone (or thyrotropin), thyrotropin-releasing hormone, or vasoactive intestinal peptide.

### Therapeutic Antibody Agents

In still other embodiments, a chitosan microbead comprises an antibody. Therapeutic antibodies can be useful for the treatment of disease and can be used as a therapeutic agent alone or in combination with other therapeutic agents as part of the therapeutic agent delivery system as described herein. Exemplary therapeutic antibodies are described by U.S. Pat. Nos. 9,333,255, 9,061,073, 8,975,377, 8,999,335, 8,883,760, 8,877,187, 8,663,950, 8,653,242, 8,486,406, 8,124,107, 8,029,785, U.S. Patent Application Nos. 20150064199, 20140328841 and International Patent Application Nos. WO 2016034968, WO 2014153056, WO 2013100120, WO 2008093331, WO 2005072479, which are incorporated herein by reference in their entirety.

### Delivery of Agents Via Chitosan-PEGDMA Compositions

The invention provides a simple means for delivering biologically active agents (e.g., small compounds, nucleic acid molecules, polypeptides) using a chitosan-PEGDMA composition. The chitosan-PEGDMA composition is delivered to a subject and the biologically active agent is eluted from the composition in situ. The chitosan-PEGDMA composition is capable of delivering a therapeutic for the treatment of a disease or disorder that requires controlled and/or localized therapeutic agent delivery over some period of time (e.g., 1, 3, 5, 7 days; 2, 3, 4 weeks; 1, 2, 3, 6, 12 months). Desirably, the chitosan-PEGDMA composition comprises an effective amount of one or more analgesics, angiogenic agents, antimicrobials, antibodies, antifungals, anti-inflammatories, anti-thrombotics, chemotherapeutics, growth factors, hormones, or steroids.

Preferably, the chitosan microbead (e.g., chitosan-PEGDMA) composition comprises at least about 1 μg, 25 μg, 50 μg, 100 μg, 250 μg, 500 μg, 750 μg, 1 mg, 5 mg, 10 mg, 25 mg, 50 mg, 75 mg, 100 mg, 200 mg, 250 mg, 300 mg, 400 mg, or 500 mg of an agent (e.g., an antimicrobial agent). In another embodiment, the composition releases at least about 1 μg, 25 μg, 50 μg, 100 μg, 250 μg, 500 μg, 750 μg, 1 mg, 5 mg, 10 mg, 25 mg, 50 mg, 75 mg, 100 mg, 200 mg, 250 mg, 300 mg, 400 mg, or 500 mg of an agent (e.g., an antimicrobial agent) over the course of at least about 1, 2, 3, 4, 5, 6, 7, 8, 9, 10, 14, 21, 28, or 35 days. In still another embodiment, the composition comprises at least about 1 jag, 25 lag, 50 jag, 100 μg, 250 μg, 500 μg, 750 μg, 1 mg, 5 mg, 10 mg, 25 mg, 50 mg, 75 mg, 100 mg, 200 mg, 250 mg, 300 mg, 400 mg, or 500 mg of an agent (e.g., an antimicrobial agent) per cm³.

### Microbeads

Crosslinking is the process which links polymer chains together. In chitosan, crosslinking induces a three-dimensional matrix of interconnected, linear, polymeric chains. The degree or extent of crosslinking depends on the crosslinking agent. Exemplary crosslinking agents include sodium tripolyphosphate, ethylene glycol diglycidyl ether, ethylene oxide, glutaraldehyde, epichlorohydrin, diisocyanate, and genipin. Crosslinking can also be accomplished using microwave or ultraviolet exposure.

Chitosan's properties can also be altered by modulating the degree of deacetylation. In one embodiment, the degree of deacetylation is adjusted between about 50-100%, wherein the bottom of the range is any integer between 50 and 99, and the top of the range is any integer between 51% and 100%. In particular embodiments, the degree of deacetylation is 51%, 55%, 60%, 61%, 65%, 70%, 71%, 73%, 74%, 75%, 76%, 77%, 78%, 79%, 80%, 85%, 90%, and 95%. In general, the higher the molecular weight, the slower the degradation of the chitosan-PEGDMA composition.

If desired, chitosan is neutralized after acid treatment. Any base known in the art (e.g., NaOH, KOH, NH₄OH, Ca(OH)₂, Mg(OH)₂, or combinations thereof) may be used to neutralize an acid-treated chitosan-PEGDMA composition. Preferably, a neutralization solution has a pH greater than 7.4 (e.g., 7.8, 8.0, 8.5, 9.0, 10, 11, and 12, 13, 14, 15, 16). The neutralization step is optional, and not strictly required. If desired, the chitosan is treated with water, PBS, or sterile saline following acid treatment. It may comprise 0.01-10.0 M of a base (e.g., 0.01, 0.025, 0.5, 0.75, 0.1, 0.25, 0.5, 0.75, 1.0, 1.5, 2.0, 2.5, 3, 4, 5, 6, 7, 8, 9, 10 M) (e.g., NaOH). Chitosan-PEGDMA compositions neutralized in bases having lower molarity degrade more quickly. Chitosan-PEGDMA compositions neutralized in bases of increased molarity degrade more slowly than those neutralized at lesser molarities. Thus, the degradation properties of chitosan can be modulated by altering the molarity of the neutralizing base.

In other embodiments, the concentration of the acidic solvent used to dissolve the chitosan is adjusted or the time period used to dissolve the chitosan is altered. For example, a 0.1%, 0.5%, 1%, 2%, 3% or 5% acid solution is used. In particular embodiments, chitosan is dissolved in acetic, citric, oxalic, proprionic, ascorbic, hydrochloric, formic, salicylic and/or lactic acids, or a combination of those. In general, acidic solvents comprising increased levels of lactic acid form chitosan-PEGDMA compositions that degrade more quickly and also have reduced strength and durability. In various embodiments, a combination of acetic and lactic acids are used. Lactic/acetic acid combinations degrade slower and are stronger. The acetic acid sponges degrade faster and are more flexible.

In contrast, lactic acid provides more flexibility. In one approach, the ratio of lactic to acetic acid is varied from 5:1, 4:1, 3:1, 2:1, 1:1, 1:2, 1:3, 1:4, to 1:5. In one embodiment, the blended acid solvent comprises 90%/10%, 80%/20% 75%/25%, 70%/30%, 60%/40%, 50%/50%. In still other embodiments, the chitosan weight % is altered from 0.25-10.0% (e.g., 0.1, 0.2, 0.25, 0.3, 0.4, 0.5, 1, 1.25, 1.5, 1.75, 2.0, 2.5, 3.0, 3.5, 4, 5, 6, 7, 8, 9, 10%). In one embodiment, a 1 wt % chitosan solution is preferred, where a 1 wt % chitosan solution contains 1 gram of chitosan per 100 ml solution. Typically, the higher the wt %, the slower the degradation.

If desired a chitosan-PEGDMA composition is loaded with agents and the chitosan-PEGDMA composition is delivered to a wound to form a delivery system for the agent. Preferably, the chitosan-PEGDMA composition contains an effective amount of a chemical or pharmaceutically active component. In one embodiment, the chitosan-PEGDMA composition self-adheres to a site at which delivery is desired. In another embodiment, an adhesive or other adhering means may be applied to the outer edges of the chitosan-PEGDMA composition to hold the composition in position during the delivery of the chemical or pharmaceutically active component. Such adherent means may be used alone or in combination with the self-adhering properties of chitosan. Chitosan-PEGDMA compositions provide for the local administration of a desired amount of a therapeutic agent.

In other embodiments, the chitosan-PEGDMA composition is administered directly to an injured area. A chitosan-PEGDMA composition of the invention is administered by sprinkling, packing, implanting, inserting or applying or by any other administration means to a site of trauma (e.g., open wound, open fracture, complex wound).

### Delivery of Chitosan Microbead Compositions

Chitosan microbead compositions can be delivered by any method known to the skilled artisan. In one approach, a chitosan-PEGDMA composition is locally delivered to a site of trauma. The chitosan-PEGDMA composition is surgically implanted at a site where promotion of healing and/or treatment or prevention of infection is required. If desired, the chitosan-PEGDMA composition is administered by a clinician within a surgical suite.

### Screening Assays

As described herein, the present invention provides for the delivery of therapeutic or prophylactic agents to wounds in vivo. The invention is based in part on the discovery that therapeutic agents can be delivered using a chitosan-PEGDMA composition. To identify chitosan-PEGDMA compositions having the desired degradation and elution profiles, screening may be carried out using no more than routine methods known in the art and described herein. For example, chitosan-PEGDMA compositions are loaded with one or more therapeutic agents and such compositions are subsequently compared to untreated control compositions to identify chitosan-PEGDMA compositions that promote healing. In another embodiment, the degradation of a chitosan-PEGDMA composition of the invention is assayed in vivo to identify the degree of deacetylation that corresponds to the desired degradation profile. Any number of methods are available for carrying out screening assays to identify such compositions.

In one working example, candidate compounds are added at varying concentrations to a chitosan microbead (e.g., chitosan-PEGDMA) composition. The degree of infection or wound healing is then measured using standard methods as described herein. The degree of infection (e.g., number of bacteria) or wound healing in the presence of the compound is compared to the level measured in a control lacking the compound. A compound that enhances healing is considered useful in the invention; such a compound may be used, for example, as a therapeutic to prevent, delay, ameliorate, stabilize, or treat a disease described herein (e.g., tissue damage). In other embodiments, the compound prevents, delays, ameliorates, stabilizes, or treats a disease or disorder described herein. Such therapeutic compounds are useful in vivo.

In another approach, chitosan microbead (e.g., chitosan-PEGDMA) compositions having varying degrees of deacetylation are incubated in vivo, added to a wound, or are contacted with a composition comprising an enzyme having chitosan-degrading activity. The length of time required for chitosan degradation is then measured using standard methods as described herein. A chitosan-PEGDMA composition having the desired degradation profile (e.g., degrading in 3 days, 5 days, 1 week, 2 weeks, 3 weeks, 1 month, 2 months, 3 months) is considered useful in the invention; such a composition may be used, for example, as a therapeutic to prevent, delay, ameliorate, stabilize, or treat a disease described herein (e.g., tissue damage). In other embodiments, the composition prevents, delays, ameliorates, stabilizes, or treats a disease or disorder described herein. Such therapeutic compositions are useful in vivo.

The present invention provides methods of treating pathogen infections (e.g., bacterial, viral, fungal), complex wounds, open fractures, trauma, and associated diseases and/or disorders or symptoms thereof which comprise administering a therapeutically effective amount of a composition comprising chitosan and a therapeutic or prophylactic agent of a formulae herein to a subject (e.g., a mammal, such as a human). Thus, one embodiment is a method of treating a subject suffering from or susceptible to an infection, trauma, wound, open fracture, or related disease or disorder that requires targeting of a therapeutic composition to a site. The method includes the step of administering to the mammal a therapeutic amount of a compound herein sufficient to treat the disease or disorder or symptom thereof, under conditions such that the disease or disorder is treated.

The methods herein include administering to the subject (including a subject identified as in need of such treatment) an effective amount of a compound described herein, or a composition described herein to produce such effect. Identifying a subject in need of such treatment can be in the judgment of a subject or a health care professional and can be subjective (e.g. opinion) or objective (e.g. measurable by a test or diagnostic method).

The therapeutic methods of the invention (which include prophylactic treatment) in general comprise administration of a therapeutically effective amount of the compounds herein, such as a compound of the formulae herein to a subject (e.g., animal, human) in need thereof, including a mammal, particularly a human. Such treatment will be suitably administered to subjects, particularly humans, suffering from, having, susceptible to, or at risk for an infection, in need of healing, having a trauma, wound, open fracture, or related disease, disorder, or symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like). The agents herein may be also used in the treatment of any other disorders in which it is desirable to promote healing or treat or prevent an infection.

In one embodiment, the invention provides a method of monitoring treatment progress. The method includes the step of determining a level of diagnostic marker (Marker) (e.g., wound healing parameters, number of bacterial cells, or any target delineated herein modulated by a compound herein, C-reactive protein, cytokine levels, or indicator thereof, etc.) or diagnostic measurement (e.g., screen, assay) in a subject suffering from or susceptible to an infection, disorder or symptoms thereof, in which the subject has been administered a therapeutic amount of a chitosan-PEGDMA composition (e.g., a chitosan-PEGDMA composition comprising a therapeutic or prophylactic agent) herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

### Test Compounds and Extracts

In general, therapeutic compounds suitable for delivery from a chitosan microbead (e.g., chitosan-PEGDMA) composition are known in the art or are identified from large libraries of both natural product or synthetic (or semi-synthetic) extracts or chemical libraries or from polypeptide or nucleic acid libraries, according to methods known in the art. Those skilled in the field of drug discovery and development will understand that the precise source of test extracts or compounds is not critical to the screening procedure(s) of the invention. Compounds used in screens may include known compounds (for example, known therapeutics used for other diseases or disorders). Alternatively, virtually any number of unknown chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds.

Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, chemical compounds to be used as candidate compounds can be synthesized from readily available starting materials using standard synthetic techniques and methodologies known to those of ordinary skill in the art. Synthetic chemistry transformations and protecting group methodologies (protection and deprotection) useful in synthesizing the compounds identified by the methods described herein are known in the art and include, for example, those such as described in R. Larock, Comprehensive Organic Transformations, VCH Publishers (1989); T. W. Greene and P. G. M. Wuts, Protective Groups in Organic Synthesis, 2nd ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Examples of methods for the synthesis of molecular libraries can be found in the art, for example in: DeWitt et al., Proc. Natl. Acad. Sci. U.S.A. 90:6909, 1993; Erb et al., Proc. Natl. Acad. Sci. USA 91:11422, 1994; Zuckermann et al., J. Med. Chem. 37:2678, 1994; Cho et al., Science 261:1303, 1993; Carrell et al., Angew. Chem. Int. Ed. Engl. 33:2059, 1994; Carell et al., Angew. Chem. Int. Ed. Engl. 33:2061, 1994; and Gallop et al., J. Med. Chem. 37:1233, 1994. Furthermore, if desired, any library or compound is readily modified using standard chemical, physical, or biochemical methods.

Libraries of compounds may be presented in solution (e.g., Houghten, Biotechniques 13:412-421, 1992), or on beads (Lam, Nature 354:82-84, 1991), chips (Fodor, Nature 364:555-556, 1993), bacteria (Ladner, U.S. Pat. No. 5,223,409), spores (Ladner U.S. Pat. No. 5,223,409), plasmids (Cull et al., Proc Natl Acad Sci USA 89:1865-1869, 1992) or on phage (Scott and Smith, Science 249:386-390, 1990; Devlin, Science 249:404-406, 1990; Cwirla et al. Proc. Natl. Acad. Sci. 87:63786382, 1990; Felici, J. Mol. Biol. 222:301-310, 1991; Ladner supra.).

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their activity should be employed whenever possible.

When a crude extract is identified as containing a compound of interest, further fractionation of the positive lead extract is necessary to isolate chemical constituents responsible for the observed effect. Thus, the goal of the extraction, fractionation, and purification process is the careful characterization and identification of a chemical entity within the crude extract that achieves a desired biological effect. Methods of fractionation and purification of such heterogeneous extracts are known in the art.

Small molecules of the invention preferably have a molecular weight below 2,000 Daltons, more preferably between 300 and 1,000 Daltons, and most preferably between 400 and 700 Daltons. It is preferred that these small molecules are organic molecules.

Chitosan microbeads comprise an effective amount of an agent described herein. Actual dosage levels and time course of administration of the active ingredients in the pharmaceutical compositions of this invention may be varied so as to obtain an amount of the active ingredient which is effective to achieve the desired therapeutic response for a particular patient, composition, and mode of administration, without being toxic to the patient. An exemplary dose range is from about 0.1 μg to 20 milligram per kilogram of body weight per day (mg/kg/day) (e.g., 0.1m/kg to 10 mg/kg, 0.1-10m/kg, 0.1-1 mg/kg). In other embodiments, the amount varies from about 0.1 mg/kg/day to about 100 mg/kg/day. In still other embodiments, the amount varies from about 0.001m to about 100m/kg (e.g., of body weight). Ranges intermediate to the above-recited values are also intended to be part of the invention.

### Kits

The invention provides kits that include chitosan microbeads (e.g., chitosan-PEGDMA) compositions. In one embodiment, the kit includes chitosan microbeads containing one or more therapeutic or prophylactic agents that prevent or treat infection or that promote healing (e.g. one or more of analgesics, angiogenic agents, antimicrobial, antibodies, antifungals, anti-inflammatories, anti-thrombotics, chemotherapeutics, growth factors, and hormones, steroid). If desired, the aforementioned chitosan microbeads (e.g., chitosan-PEGDMA) compositions further comprise an agent described herein.

In some embodiments, the kit comprises a sterile container which contains a chitosan microbead (e.g., chitosan-PEGDMA) composition; such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

If desired a chitosan microbead (e.g., chitosan-PEGDMA) composition of the invention is provided together with instructions for using it in a prophylactic or therapeutic method described herein. The instructions will generally include information about the use of the composition for the treatment of a trauma, infection or related disease in a subject in need thereof. The instructions may be printed directly on the container (when present), or as a label applied to the container, or as a separate sheet, pamphlet, card, or folder supplied in or with the container.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: Magnetic Nanoparticle (MNP) Characterization

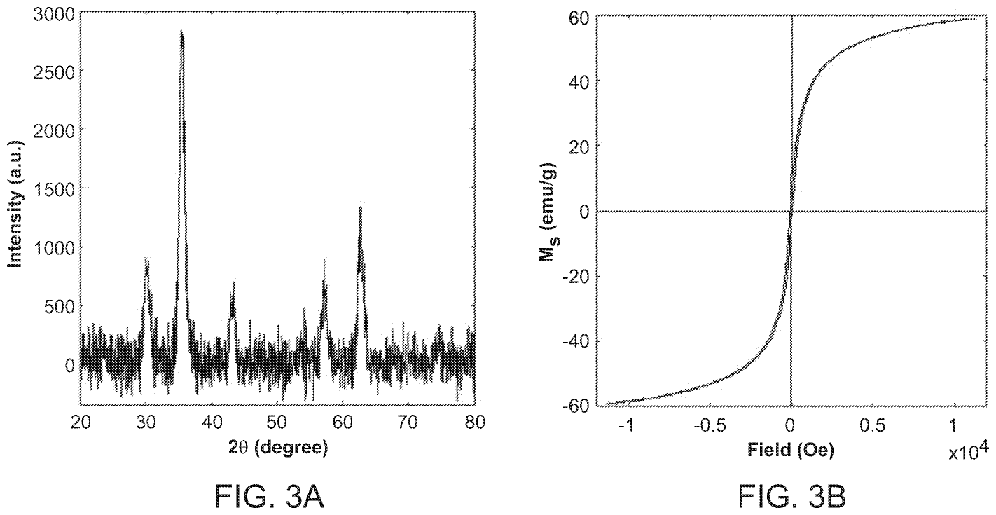

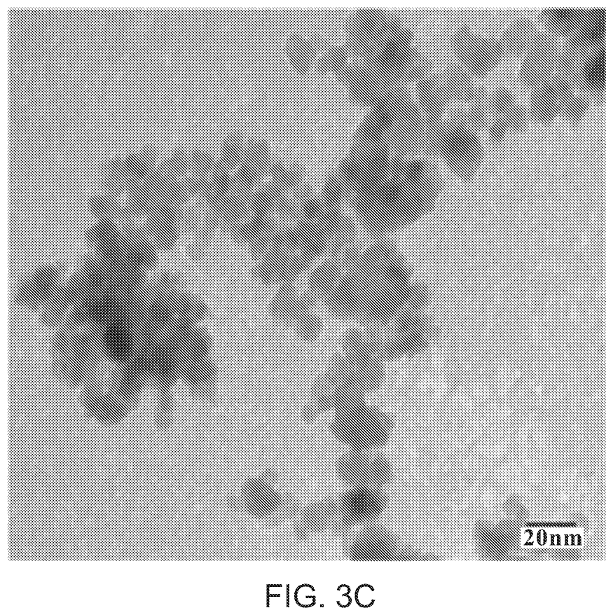

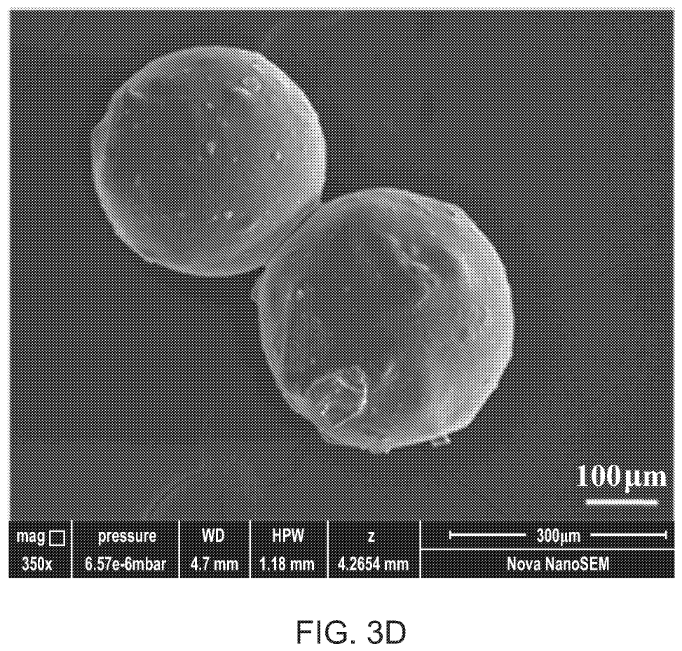

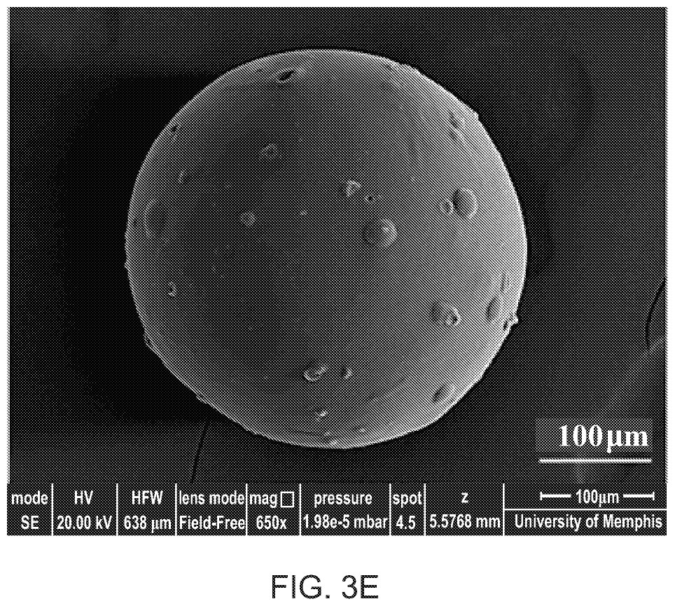

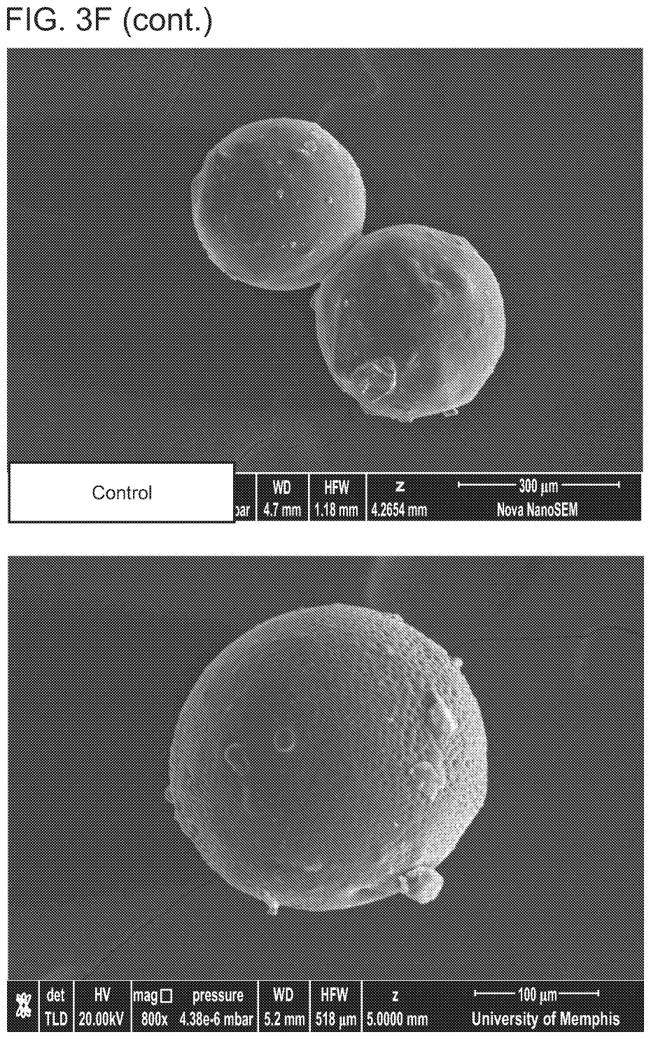

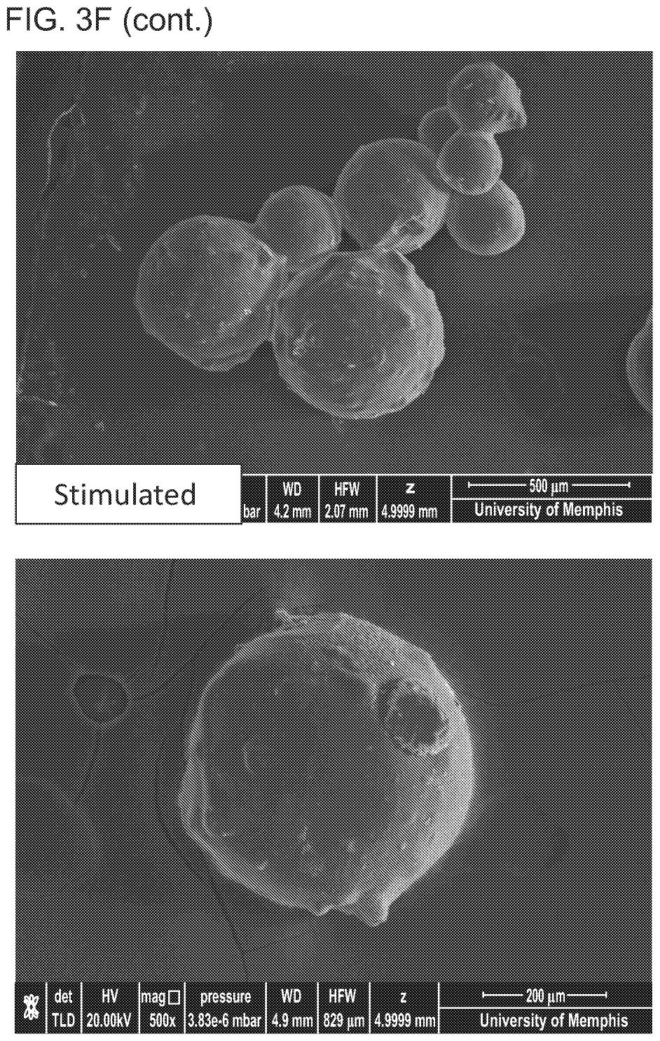

To characterize the physical properties of the Magnetic Nanoparticles (MNPs) a series of test were conducted. The X-Ray Diffraction (XRD) curve (FIG. 3A) and magnetization curve (FIG. 3B) of the MNP were similar to the data reported in other literature [43, 44]. The MNPs were imaged by Transmission Electron Microscopy (TEM) (FIG. 3C) and the size distribution was calculated to be 10.89±2.67 nm.

### Example 2: Chitosan Microbead Characterization

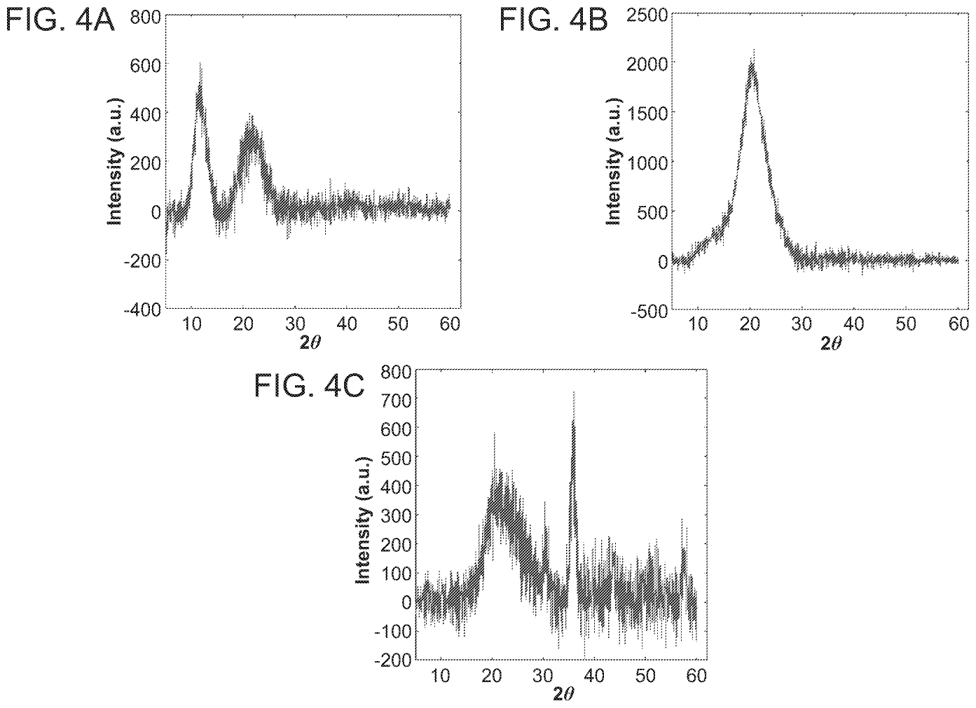

To characterize the physical properties of the chitosan microbeads a series of test were conducted. The XRD of vancomycin and chitosan microbeads with/without MNP are shown in FIGS. 4A-4C. XRD plots are shown for vancomycin (FIG. 4A), chitosan with PEGDMA and vancomycin (FIG. 4B), chitosan with PEGDMA, vancomycin and MNP (FIG. 4C). The major peaks of chitosan, vancomycin and MNP (FIG. 4C) support the presence of these constituents in the final microbeads. The microbeads were imaged using a Scanning Electron Microscope (SEM) (FIG. 3D) and the size distribution of these particles was 288.4±62.2 μm. SEM images of chitosan microbeads with MNP are provided in FIG. 3D and without MNP in FIG. 3E. SEM imaged of stimulated and control MNP-loaded microbeads at various magnifications are shown in FIG. 3F, showing that integrity of bead structure is not significantly changed after stimulation.

### Example 3: Analysis of Stimulus Parameters

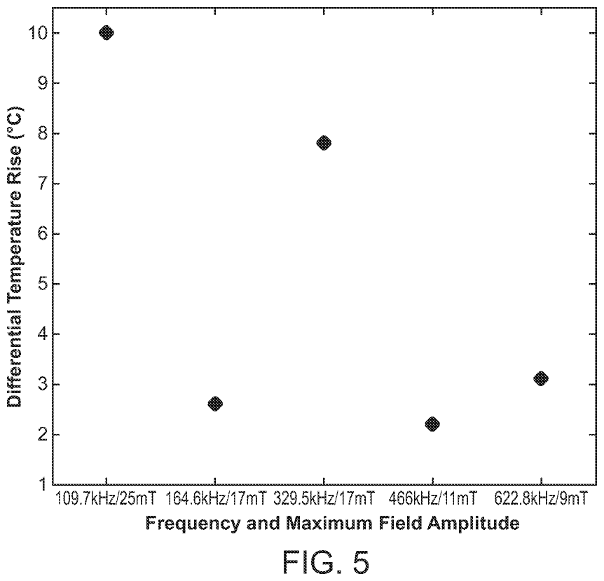

Tests were conducted to determine and analyze the stimulus parameters. The differential increase in temperature under different pre-determined frequency/magnetic field intensity combinations for the same duration and sample is shown in FIG. 5. For a frequency of 109.7 kHz and a magnetic field intensity of 25 mT the highest temperature increment of 10° C. was recorded and therefore this combination was utilized for all stimulation tests.

### Example 4. Stimulation of Chitosan Microbeads with Magnetic Nanoparticles Results in a Statistically Significant Drug Increase

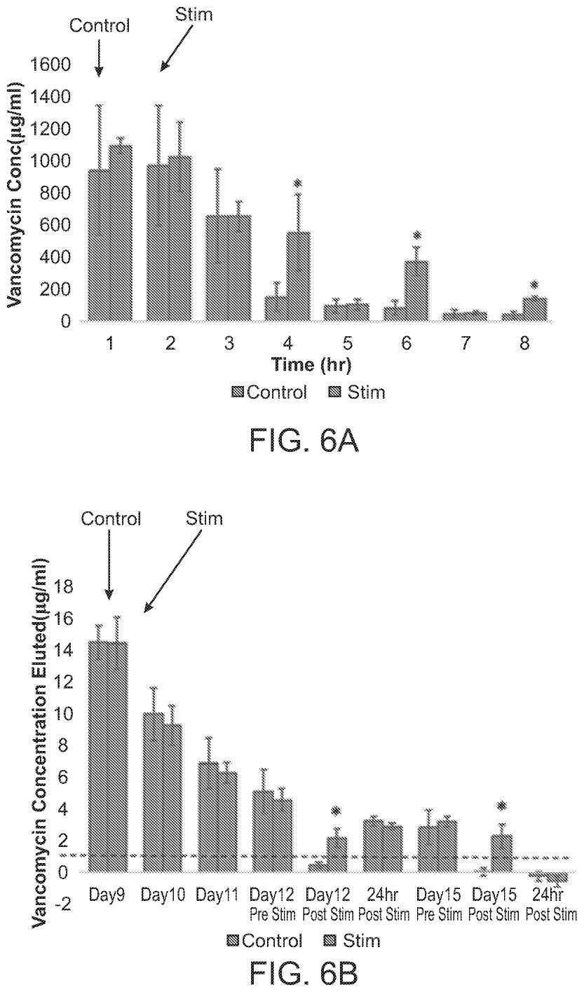

A short term elution study was conducted. In these stimulation spans, it was observed that the temperature rose from an initial 22° C. to 38° C. The concentration was graphed as bar-plot (FIG. 6A), which shows a statistically significant drug increase by stimulated samples. After stimulation at each of the 3 instances, the test groups released a higher amount of vancomycin compared to control, with statistically measured p-values of 0.008, 0.008 and 0.008 respectively. Assuming a p<0.05 as a significant difference, the recorded p-values are conclusive of a higher amount of vancomycin being eluted by the samples post-stimulation. In the periods when no stimulation was given, the test groups seemed to release as much drug as the control. This was confirmed by p-values of 1, 0.84, 1, 0.69, 0.84 calculated for vancomycin eluted at t=1, 2, 3, 5, 7 hr respectively between both groups.

A long term elution study was conducted. The study showed that by stimulating the test groups, the vancomycin elution was increased above the theoretically effective minimum inhibitory values against Staphylococcus aureus in the stimulation period (FIG. 6B). On both instances of stimulation of Day 12 and Day 15, the test groups released statistically significant higher amounts of vancomycin, calculated at p=0.002, 0.002 respectively. In the non-stimulation periods, there was no detectable differences in therapeutic agent elution between both groups, confirmed by p-values of 0.7, 0.7, 0.94, 0.59, 0.13, 0.82, 0.06 on Day 9, 10, 11, 12 pre-stimulus, 24 hr post-stimulus on 12, 15 pre-stimulus and 24 hr post-stimulus on 15.

### Example 5. Experiments on Chitosan Microbeads

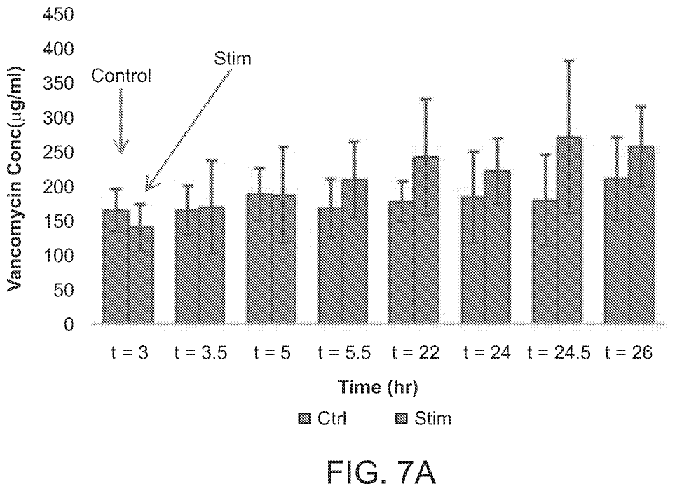

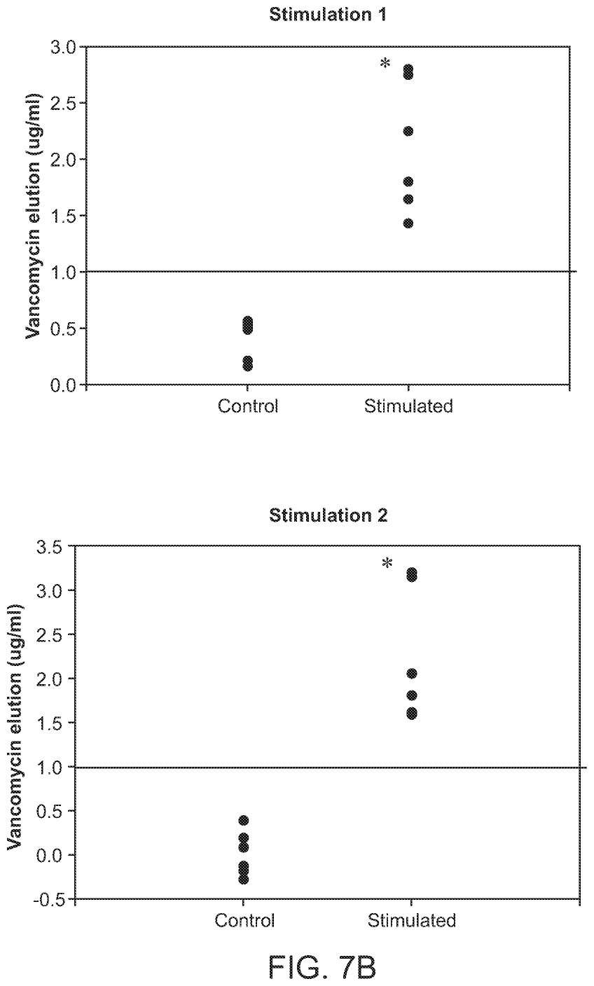

The vancomycin concentration detected at each sampling points is represented as a bar plot in FIG. 7A. Non-MNP loaded beads eluted similar amounts of drug over the course of 24 hours. Stimulus was a magnetic field (109.9 kHz, 25 mT) applied for 30 min to test tubes containing 10 mg beads/ml PBS at 3, 5 and 24 hours. Amount of drug release in the hours after stimulation was not significantly different between control (non-stimulated) and experimental (stimulated) samples, demonstrating that the chitosan microbeads were not responsive to magnetic stimulation and the chitosan microbeads were not the cause for greater drug release compared to the controls. Data represented is average±standard deviation (n=5). The p-values of the therapeutic agent elution difference between both groups were >0.05, implying that stimulating the test groups did not initiate them to release the drug more than the control groups.

The concentration of vancomycin released over time with and without stimulation on MNP loaded beads is shown in FIG. 6C. Stimulation significantly increased the amount of vancomycin released from MNP loaded beads in stimulation 1 and stimulation 2. The lines represent effective minimum inhibitory values for vancomycin against S. aureus. In the absence of stimulation, a significant release of vancomycin was not observed. Data points represent individual test values. Asterisks represent statistically significant differences between stimulated and control groups, p<0.05. The data present in FIGS. 7A and 7B demonstrates that magnetic nanoparticles are vital for assisting in drug release by magnetic hyperthermia.

In conclusion, a novel therapeutic agent delivery system composed of chitosan, cross-linking polymer, magnetic nanoparticles, and loaded with vancomycin was successfully tested in vitro as being positively responsive to external stimuli that caused an elevation in temperature. Magnetic excitation was chosen over other stimulation modalities because it does not need any physical contact with the patient, is considered bio-safe, and its parameters can be quantified accurately. The data presented herein demonstrated that the therapeutic agent delivery system responded well to stimulus by discharging significant amount of drug compared to control samples. The experiments presented herein further indicate that the therapeutic agent delivery system of the present invention has the potential to burst-release higher amount of drugs on multiple instances of stimulus, several hours or days apart as needed, and thus might enable maintenance of MIC levels in avascular areas. The therapeutic agent delivery system can aid in targeting drug directly to problem areas, preventing systemic toxicity. Furthermore, since the therapeutic agent delivery system responded to a static magnetic field as well, the therapeutic agent delivery system has the capability to be guided, localized and confined at the target site, from where the drug can be released when required by an external alternating magnetic field. The therapeutic agent delivery system also has the potential of enhancing MRI contrast [16-20]. These features would greatly assist clinicians in controlling therapeutic agent delivery, dosage timings and strength as the needs of the patient dictate.

### Example 6: In Vivo Analysis of Drug Release

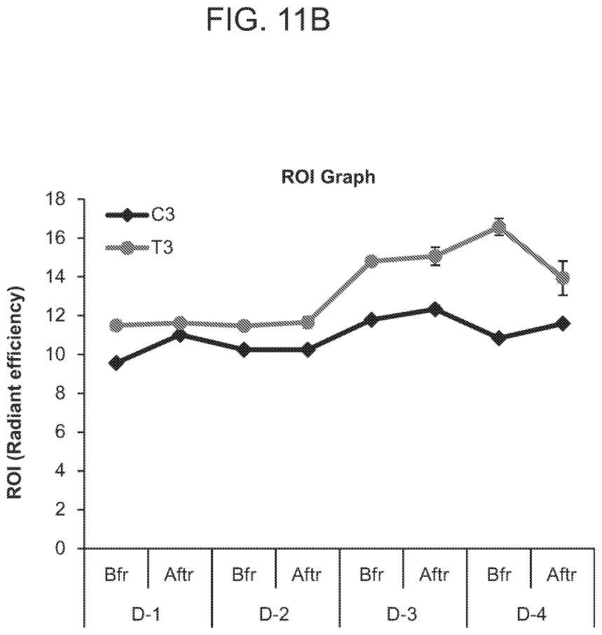

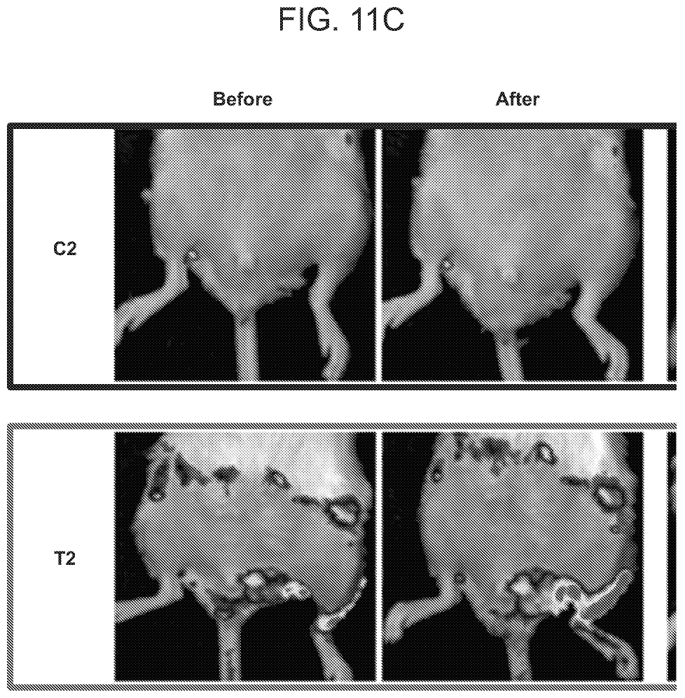

MNP-loaded chitosan beads containing rhodamine, as a model therapeutic drug molecule, were injected into the mouse leg muscle tissue of mice. Microbeads suspended in glycerol were delivered through a 16 gauge needle. Imaging with a fluorescence camera (In vivo imaging system, IVIS) was performed immediately after injection, as well as before and after magnetic stimulation. Microbeads containing MNP did not fluoresce alone or immediately after implantation, in contrast with non-MNP microbeads. Fluorescence in the region of interest (ROI), of tissue surrounding the microbeads was observed due to diffusion of drug into tissue. Magnetic stimulations occurred on day 1, day 2, day 3, and day 4 (FIG. 11A). FIG. 11A shows the treatment schedule and FIG. 11B shows results for one matched set (control vs. treatment with magnetic stimulation). FIGS. 11B and 11C indicate that more drug was released in stimulated groups than in control groups after day 2. Histological scoring of inflammatory response showed no statistical difference between stimulated and non-stimulated animals in inflammatory response. Histological sections were reviewed and scored by a pathologist using a rubric based on reaction zone and the density and types of cells present around implanted microbeads to assess inflammatory tissue.

The results described herein above, were obtained using the following methods and materials.

### Preparation of Magnetic Nanoparticles (MNPs)

Monodisperse MNPs of iron oxide (Fe3O4) were formed by reacting Iron(II) chloride (FeCl2) with Iron(III) chloride (FeCl3) at a molar ratio of 0.5 dissolved in hydrochloric acid (HCl) by dropping into a basic solution of sodium hydroxide (NaOH) between pH 11 and 12.1 MNPs were washed with HCl and deionized water several times.

Kang et al. (Chem. Mater. 8, pp. 2209-2211) discusses a non-surfactants method of generating Fe3O4 MNPs. The MNPs were then imaged by a Transmission Electron Microscope (TEM) and the sizes of 500 individual particles were measured with Image.” Average nanoparticle size was 12 nm. D8/Advance (Bruker Advance X-Ray Solutions) was used to measure X-Ray Diffraction (XRD). A magnetization curve for the same samples was obtained by VSM-130 (Dexing Magnet Tech. Co.).

### Preparation of Chitosan Microbeads

The preparation of the chitosan component of the therapeutic agent delivery system can be divided into three phases; first being the phase where chitosan solution is made, the second is the emulsification process and the last is the washing stage.

On day 1, a solution of 4% wt. chitosan, 2% wt. MNP (Fe3O4), 1% volume glacial acetic acid and 0.8% vancomycin was made. 1 g MNP was added to 46 ml DI water in a 50 ml centrifuge tube. The mixture was vortexed then sonicated for 1 hour. The mixture was then added to 2 g chitosan (Chitopharm S) and 0.4 g vancomycin (MP Biomedicals). 0.5 ml glacial acidic acid was added and the solution was stirred well. The solution was set up on an overhead impeller and left to stir overnight.

On day 2, 2 g Span 80 surfactant (Sigma Aldrich), 75 ml light mineral oil, 75 ml heavy mineral oil (Fisher Scientific) and 15 ml polyethylene dimethacrylate (PEGDMA) Mn=550 (Sigma Aldrich) were combined in a 400 ml beaker. This solution was placed on a hot plate and stirred by an overhead impeller. Speed was again set at the highest setting possible without inducing splashing. 15 ml chitosan solution from day one was loaded into a 30 ml syringe and quickly injected into the stirring oil, span 80 and PEGDMA mixture. The hot plate was set to 60° C. and left for 24 hours.

The day 2 mixture was removed from the impeller. After draining the excess oil, the beads and remaining oil was then poured into a 50 ml centrifuge tube and centrifuged for 12 minutes at 330 g to pack the beads at the bottom of the tube. The supernatant was poured off. The tube was filled with approximately 30 ml hexanes (Fisher) and vortexed. The tube was centrifuged for 10 min at 330 g, then the hexanes were poured off. This process was repeated 2 more times with hexanes, once with methanol and once with acetone, all with 8 min centrifuge times at 330 g. After the final wash, the beads were re-suspended in approximately 10 ml of acetone and poured into a glass petri dish to dry.

For SEM imaging, the samples were fixed on a carbon tape and sputter coated with 5 nm Au/Pd.

### Alternative Chitosan Bead Preparation Method

On the first day of chitosan microbead preparation, a solution of 4% wt. chitosan, 2% wt. MNP (Fe₃O₄), 1% volume glacial acetic acid and 0.8% vancomycin was made: lg MNP was added to 46 ml DI water in a 50 ml centrifuge tube. The mixture was vortexed then sonicated for 1 hour. The mixture was then added to 2g chitosan (Chitopharm S) and 0.4g vancomycin (MP Biomedicals). 0.5 ml glacial acidic acid was then added and the solution was stirred by hand until all large clumps dissolved. The solution was set up on an overhead impeller at the fastest speed possible while avoiding splashing and left to stir overnight. Other methods may also be used at this stage for the chitosan microbead preparation. For example, a higher percentage of acetic acid in the solution may be prepared as follows: 4 w/v % chitosan in 5% acetic acid with and without MNP dispersed via syringe into a 50:50 ratio of light:heavy chain liquid paraffin oil (Thermo Fisher Scientific, Massachusetts, USA) and stirred at 1200 rpm with a magnetic stirrer. Other methods for mixing or washing may also be used in the preceding steps, such as the use of a filtration system (e.g., addition of hexanes instead of pouring off oil, followed by the pouring off of the hexanes, centrifugation, repeat etc.).On the second day of chitosan microbead preparation an emulsification protocol was conducted. The following components were combined to form a solution in a 400 ml beaker: 2g Span 80 surfactant (Sigma Aldrich), 75 ml light mineral oil, 75 ml heavy mineral oil (Fisher), and 15 ml PEGDMA Mₙ=550 (Sigma Aldrich). This solution was placed on a hot plate and stirred by an overhead impeller. Speed was again set at the highest setting possible without inducing splashing. 15 ml chitosan solution from day one was loaded into a 30 ml syringe and quickly injected into the stirring oil, span 80 and PEGDMA mixture. The hot plate was set to 60° C. and left for 24 hours.

On the third day of chitosan microbead preparation a washing protocol was conducted. The day 2 mixture was removed from the impeller.

Excess oil was drained off and discarded. The beads were transferred into a glass microfiltration assembly driven by a vacuum pump. The beads were rinsed with hexanes, methanol and then acetone to remove residual oil and reactants. After the beads dried, they were transferred to a 15 mL centrifuge tube and stored in a desiccator.

### Procedure for Generation and Application of Stimulus

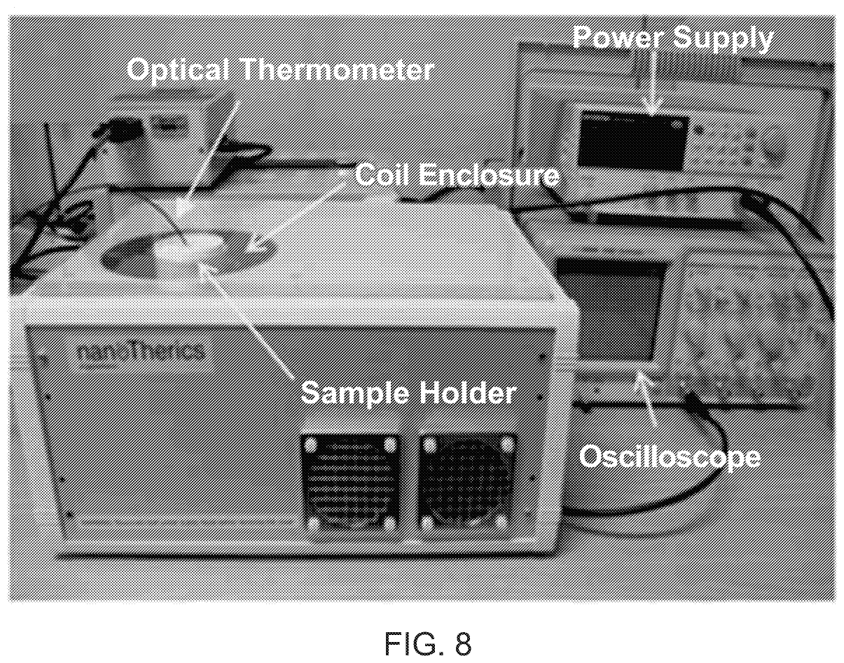

A MagneTherm instrument (nanoTherics, UK, FIG. 8) was used to provide magnetic stimulation. It consists of interchangeable 9 and 17 turn coils with 10 different capacitor banks, each of which is characterized by a specific resonant frequency and maximum magnetic flux density. The coil is water-cooled and positioned around a sample holder. A frequency of 109.9 kHz, and an amplitude of 25 mT for all experiments. A fiber optic thermometer (Optocon, Germany) was used for accurate temperature measurements of samples.

It is necessary to maximize temperature rise over the stimulation period for enhancement of drug release by hyperthermia. A test of various frequencies and intensities was performed and compared to determine the combination of frequency/intensity that caused the highest temperature rise in MNP. 2 mL of PBS was added to 185 mg of pure MNP and stimulated for 10 minutes for each of 5 frequency/intensity pairing pre-fixed and provided by the manufacturer.

Experiments on Chitosan Microbeads with Magnetic Nano Particles

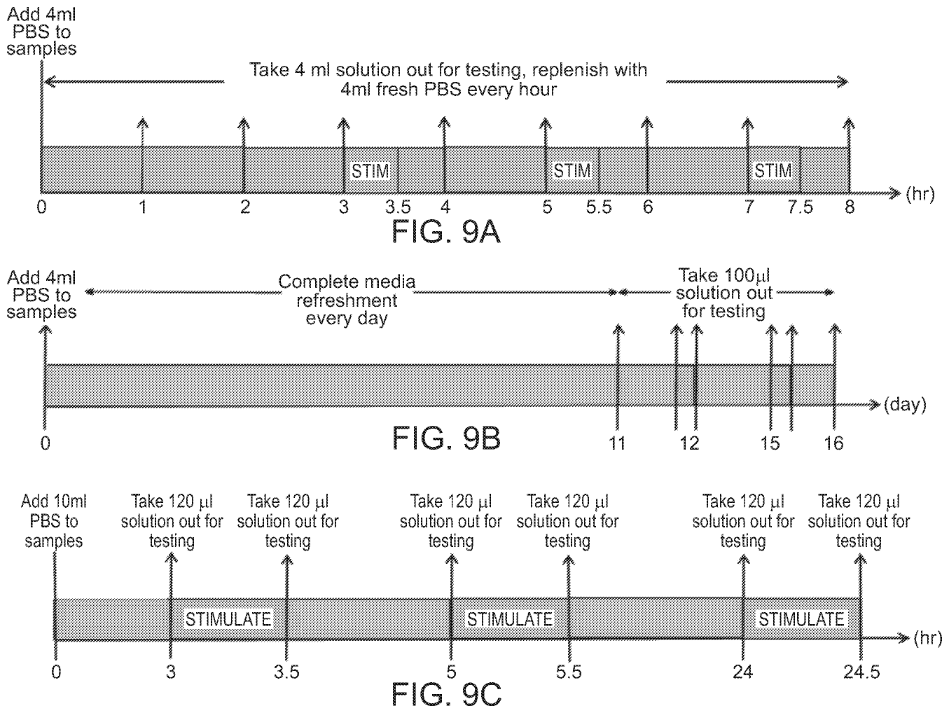

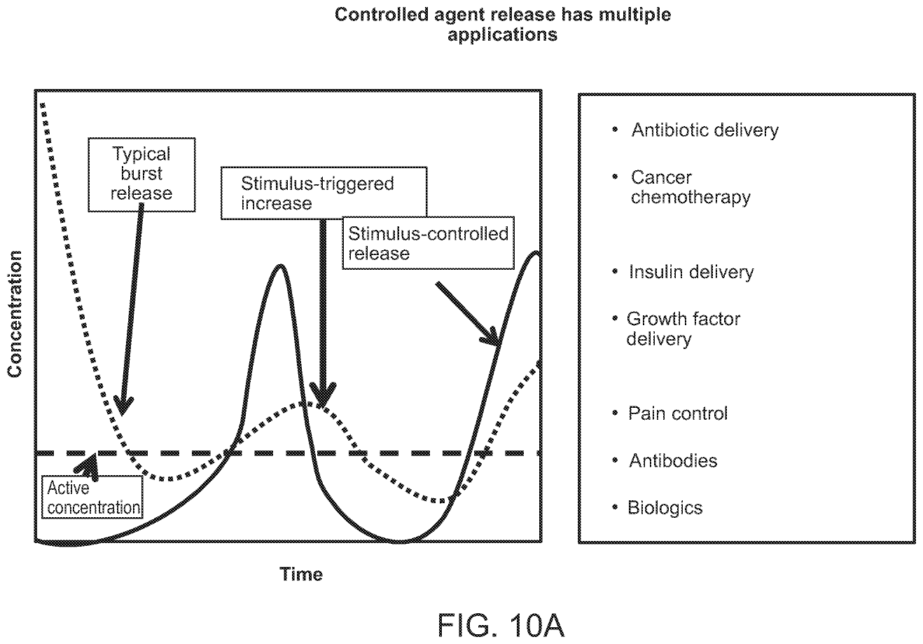

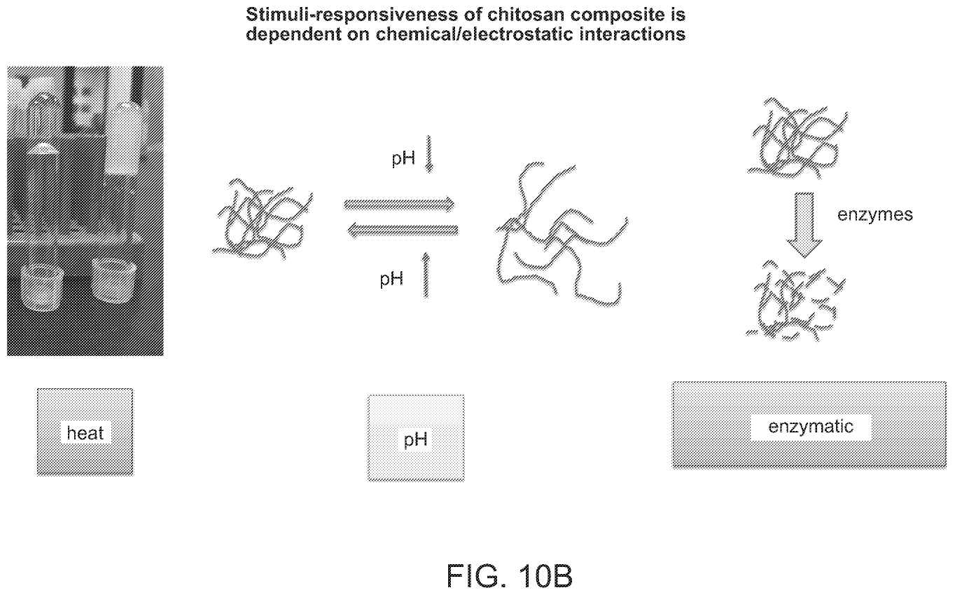

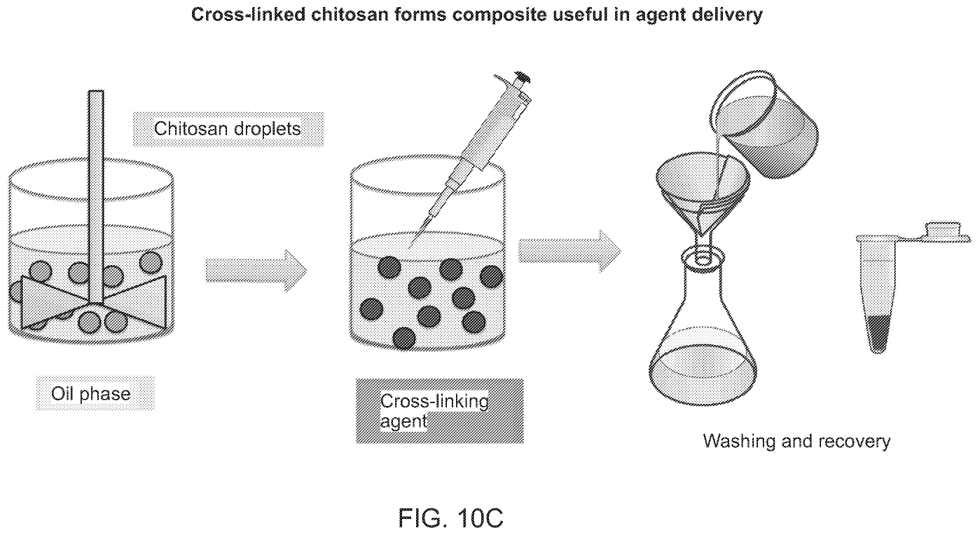

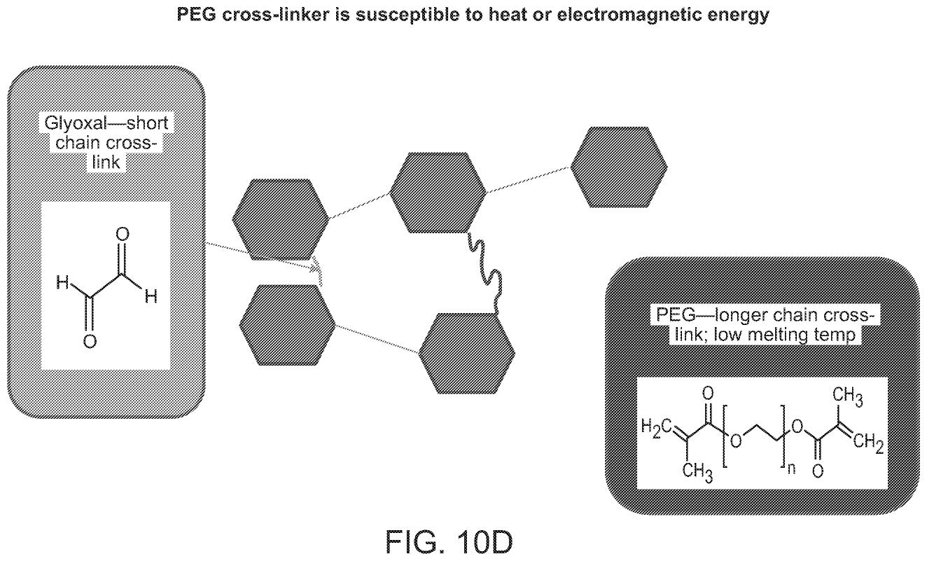

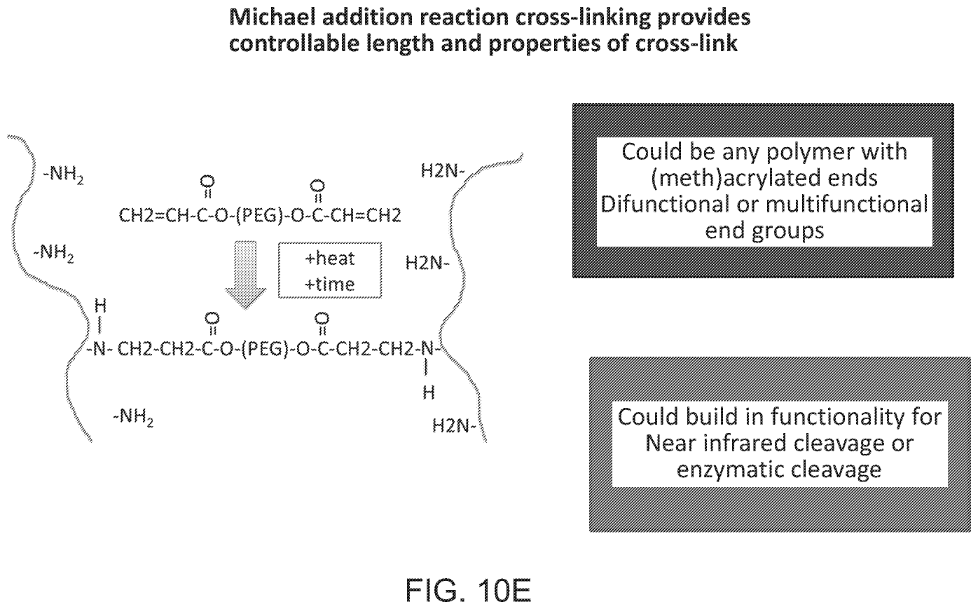

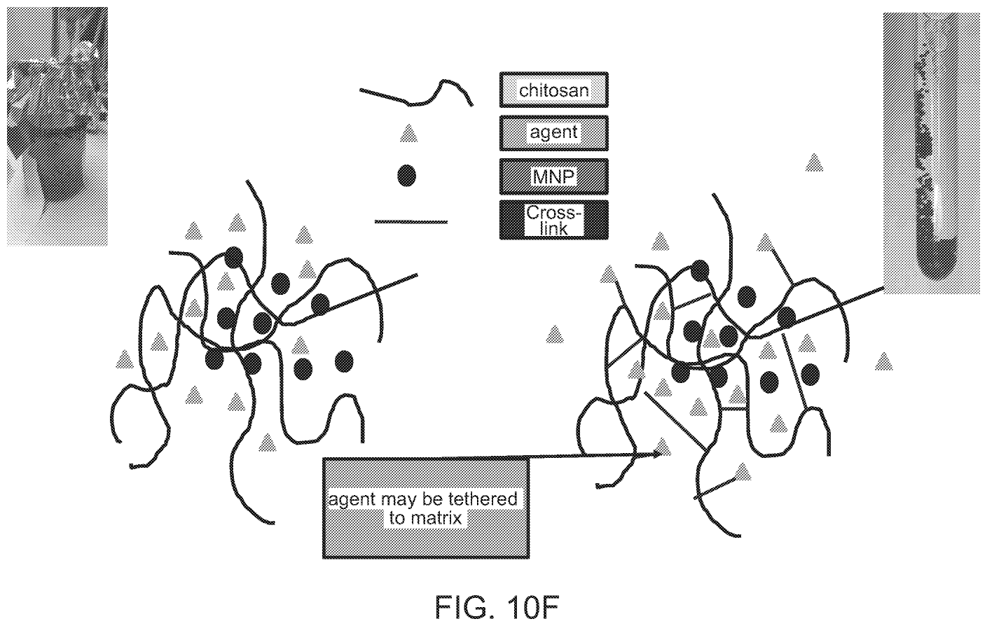

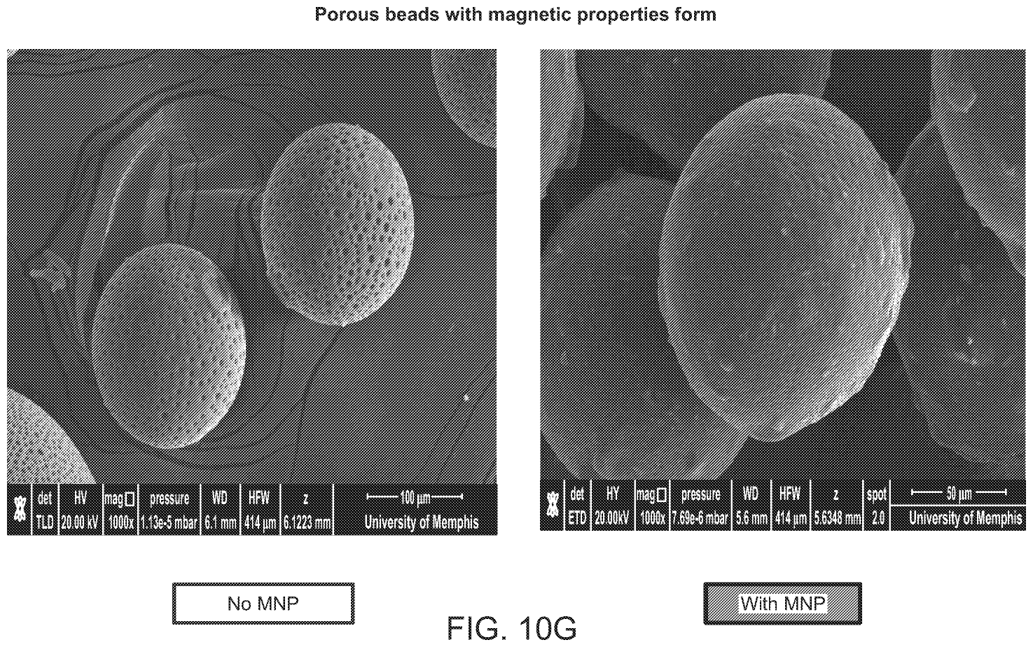

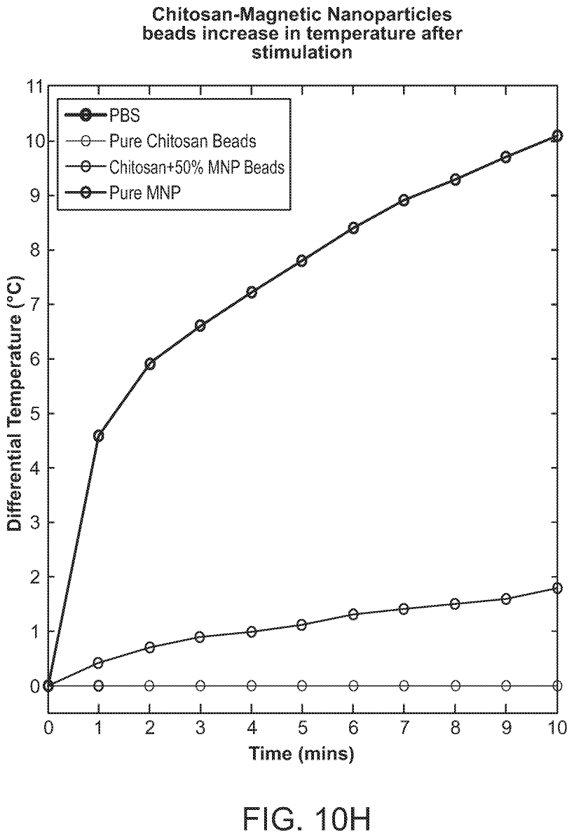

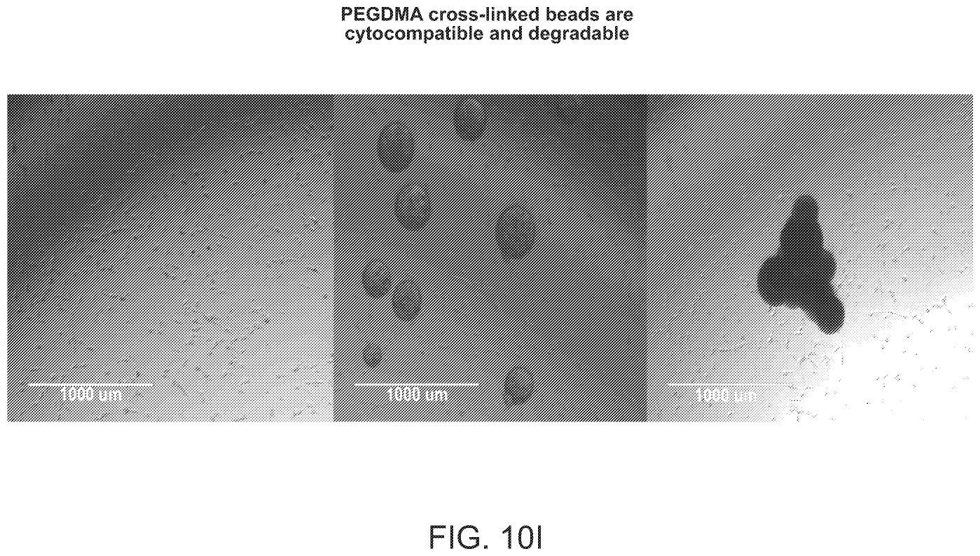

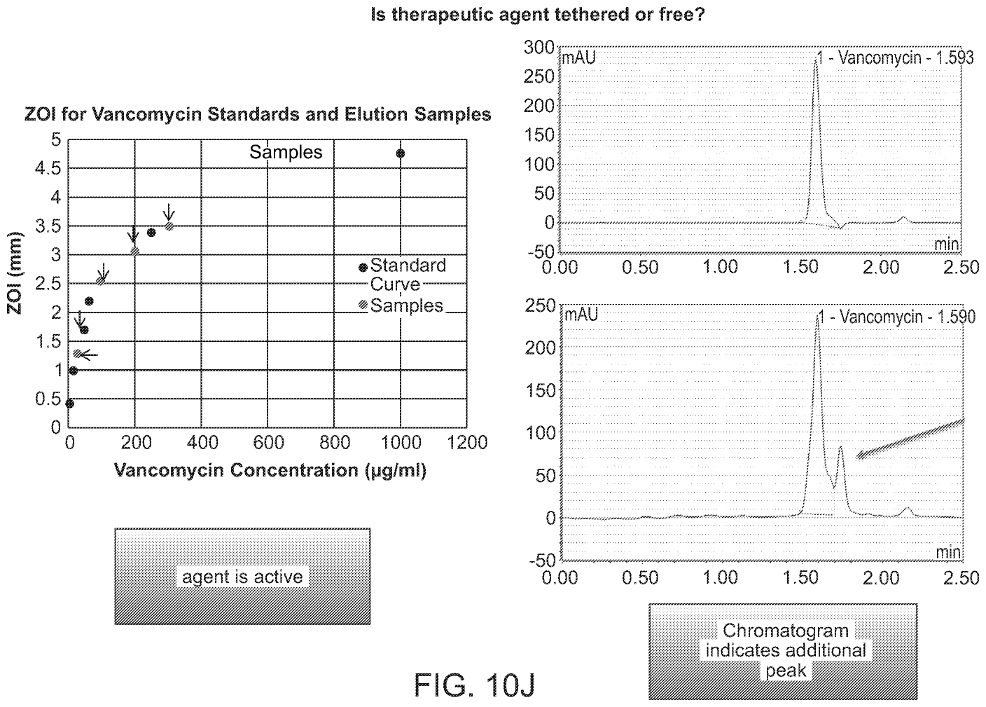

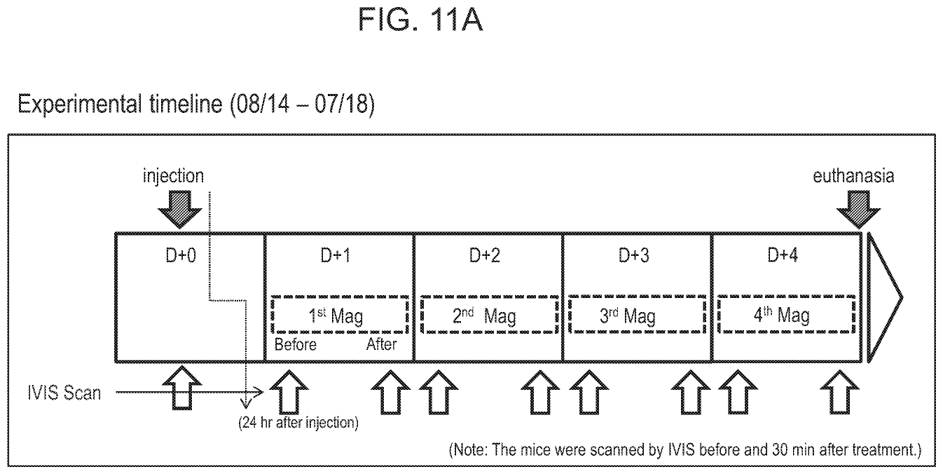

For the short term elution study batches of chitosan microbeads were divided into 10 samples of 100 mg each, of which 5 were assigned as control and 5 for magnetic hyperthermia. To each sample, 4 mL of PBS was added. The total duration of the experiments was 8 hours and is shown in FIG. 4(a). The PBS is completely refreshed with new PBS every 1 h. The test groups were stimulated at 3rd, 5th and 7th hour for 30 min as shown in FIG. 9A.

An ideal application of this therapeutic agent delivery system is to check the viability of stimulation after several days. A long-term study was carried out where the samples were stimulated on Day 12 and Day 15, depicted in FIG. 9B. The experiments comprised of 6 control and 6 test samples, each with 100 mg chitosan microbeads with MNP. 4 ml of 1×PBS was added to all of them. The media was completely refreshed up to Day 11. The media was not refreshed on Day 12 to ensure vancomycin concentrations stayed above HPLC system requirements. 100μ.1 from the PBS was collected before and after stimulation on day 12 and 15. Additional 100 μl samples were also collected on day 13 and day 16. Both stimulations were of 60 min each.

Experiments on Chitosan Microbeads without Magnetic Nanoparticles.

To check if a similar drug release could be achieved without the presence of magnetic nanoparticles.

This experiment was vital to prove the role of magnetic nanoparticles in aiding drug release. In these set of experiments, there were 5 control and 5 test samples containing 100 mg of microbeads. Due to the very fine and light nature of these beads, complete media refreshment was not possible without pipetting out several microbeads each time. To avoid introducing errors due to non-consistent sample weight, a slightly different timeline was followed, drawn in FIG. 9C. At t=0, 10 ml of PBS was added to all samples. 120Aμ.1 of the supernatant was collected only before and after the stimulation time points. Stimulation was given at 3rd, 5th and 24th hour for 30 minutes.

### Data Collection, Calibration and Analysis

High performance liquid chromatography (HPLC) was used for analyzing the amount of vancomycin released from the collected supernatant. The non-parametric Mann Whitney test was used to analyze the data. The significance level for assessing significant differences in therapeutic agent elution was fixed at 5%.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

### REFERENCES

1. A. Mohapatra, B. I. Morshed, W. O. Haggard, R. A. Smith. “Stealth Engineering for in vivo Drug Delivery Systems”. Critical Reviews in Biomedical Engineering, vol. 43, pp. 347-69, 2016.
2. K. Belfield, R. Bayston, J. P. Birchall, M. Daniel. “Do orally administered antibiotics reach concentrations in the middle ear sufficient to eradicate planktonic and biofilm bacteria? A review”. International Journal of Pediatric Otorhinolaryngology, vol. 79, pp. 296-300, 2015.
3. Y. Shi, A. Wan, Y. Shi, Y. Zhang, Y. Chen. “Experimental and Mathematical studies on the drug release properties of aspirin loaded chitosan nanoparticles”. BioMed Research International, vol. 2014, 2014.
4. R. Cheng, F. Meng, C. Deng, H. Klok, Z. Zhong, “Dual and multi-stimuli responsive polymeric nanoparticles for programmed site-specific drug delivery”. Biomaterials, vol. 34, pp. 3647-3627, 2013.
5. M. N. Yasin, D. Svirskis, A. Seyfoddin, I. D. Rupenthal, “Implants for drug delivery to the posterior segment of the eye: A focus on stimuli-responsive and tunable release systems”. Journal of Controlled Release, vol. 196, pp. 208-221, 2014.
6. C. P. McCoy, N. J. Irwin, C. Brady, D. S. Jones, G. P. Andrews, S. P. Gorman. “Synthesis and release kinetics of polymerisable ester drug conjugates: towards pH-responsive infection-resistant urinary biomaterials”. Tetrahedron Letters, vol. 54, pp. 2511-2514, 2013
7. A. E. Deatsch, B. A. Evans. “Heating efficiency in magnetic nanoparticle hyperthermia”. Journal of Magnetism and Magnetic Materials, vol. 354, pp. 163-172, 2014.
8. R. Hergt, W. Andra, C. G. d'Ambly, I. Hilger, W. A. Kaiser, U. Richter, H. Schmidt. “Physical limits of hyperthermia using magnetite fine particles”. IEEE Transactions on Magnetics, vol. 34, pp. 3745-3754, 1998.
9. R. Kötitz, W. Weitschies, L. Trahms, W. Semmler. “Investigation of Brownian and Néel relaxation in magnetic fluids”. Journal of Magnetism and Magnetic Materials, vol. 201, pp. 102-104, 1999.
10. R. Hergt, S. Dutz, R. Müller, M. Zeisberger. “Magnetic particle hyperthermia: nanoparticle magnetism and materials development for cancer therapy”. Journal of Physics: Condensed Matter, vol. 18, pp. 2919-2934, 2006.
11. G. T. Landi. “Simple models for the heating curve in magnetic hyperthermia experiments”. Journal of Magnetism and Magnetic Materials, vol. 326, pp. 14-21, 2013.
12. R. E. Rosensweig. (2002). Heating magnetic fluid with alternating magnetic field. Journal of Magnetism and Magnetic Materials. 252, pp. 370-374.
13. J. Kost, J. Wolfrum, R. Langer. “Magnetically enhanced insulin release in diabetic rats”. Journal of Biomedical Materials Research Part A, vol. 21, pp. 1367-73, 1987.
14. Q. A. Pankhurst, N. K. T. Thank, S K Jones, J. Dobson. “Progress in applications of magnetic nanoparticles in biomedicine”. Journal of Physics D: Applied Physics, vol. 42, pp. 1-15, 2009.
15. B. Chertok, A. E. David, V. C. Yang. “Polyethyleneimine-modified iron oxide nanoparticles for brain tumor drug delivery using magnetic targeting and intra-carotid administration”. Biomaterials, vol. 31, pp. 6317-6324, 2010.
16. W. Xu, K. Kattel, J. Y. Park, Y. Chang, T. J. Kim, G. H. Lee. “Paramagnetic nanoparticle T1 and T2 MRI contrast agents”. Physical Chemistry Chemical Physics, vol. 14, pp. 12687-12700, 2012.
17. S. H. Lee, B. H. Kim, H. B. Na, T. Hyeon. “Paramagnetic inorganic nanoparticles as T1 MRI contrast agents”. Wiley Interdisciplinary Reviews: Nanomedicine and Nanobiotechnology, vol. 6, pp. 196-209, 2014.
18. K. Ohno, C. Mori, T. Akashi, S. Yoshida, Y. Tago, Y. Tsujii, Y. Tabata. “Fabrication of contrast agents for magnetic resonance imaging from polymer-brush-afforded iron oxide magnetic nanoparticles prepared by surface-initiated living radical polymerization”. Biomacromolecules, pp. 3453-3462, 2013.
19. Q. L. Jiang, S. W. Zheng, R. Y. Hong, S. M. Deng, L. Guo, R. L. Hu, B. Gao, M. Huang, L. F. Cheng, G. H. Liu, Y. Q. Wang. “Folic acid-conjugated Fe3O4 magnetic nanoparticles for hyperthermia and MRI in vitro and in vivo”. Applied Surface Science, vol. 307, pp. 224-233, 2014.
20. P. Chen et al., “Novel magnetic/ultrasound focusing system enhances nanoparticle drug delivery for glioma treatment”. Neuro-Oncology, vol. 12, pp. 1050-1060, 2010.
21. S. Bucak, B. Yavurtirk, A. D. Sezer. “Magnetic nanoparticles: synthesis, surface modifications and application in drug delivery”. in Recent Advances in Novel Drug Carrier Systems, INTECH Open Access Publisher, 2012.
22. P. V. Finotelli, D. D. Silva, M. Sola-Penna, A. M. Rossi, M. Farina, L. R. Andrade, A. Y. Takeuchi, M. H. Rocha-Ledo. “Microcapsules of alginate/chitosan containing magnetic nanoparticles for controlled release of insulin”. Colloids and Surfaces B: Biointerfaces, vol. 81, pp. 206-211, 2010.
23. S. Hu, C. Tsai, C. Liao, D. Liu, S. Chen. “Controlled Rupture of Magnetic Polyelectrolyte Microcapsules for Drug Delivery”. Langmuir, vol. 24, pp. 1181-11818, 2008.
24. B. Koppolu, M. Rahimi, S. Nattama, A. Wadajkar, K. T. Nguyen. “Development of multiple-layer polymeric particles for targeted and controlled drug delivery”. Nanomedicine, vol. 6, pp. 355-361, 2010.
25. K. Katagiri, Y. Imai, K. Koumoto. “Variable on-demand release function of magnetoresponsive hybrid capsules”. Journal of Colloid and Interface Science, vol. 361, pp. 109-114, 2011.
26. A. Bernkop-Schnürch, S. Dünnhaupt. “Chitosan-based drug delivery systems”. European Journal of Pharmaceutics and Biopharmaceutics, vol. 81, pp. 463-469, 2012.
27. T. Jiang, R. James, S. G. Kumbar. C. T. Laurencin, Natural and Synthetic Biomedical Polymers, ch. 5, Elsevier, 2014, 2014.
28. Y. Luo, Q. Wang. “Recent developments of chitosan-based polyelectrolyte complexes with natural polysachharides for drug delivery”. International Journal of Biological Macromolecules, vol. 64, pp. 353-367, 2014.
29. M. P. Patel, R. R. Patel, J. K. Patel. “Chitosan Mediated Targeted Drug Delivery System: A Review”. Journal of Pharmaceutical Sciences, vol. 13, pp. 536-557, 2010.
30. R. M. Patil, P. B. Shete, N. D. Thorat, S. V. Otari, K. C. Barick, A. Prasad, R. S. Ningthoujam, B. M. Tiwale, S. H. Pawar. “Superparamagnetic iron oxide/chitosan core/shells for hyperthermia application: Improved colloidal stability and biocompatibility”. Journal of Magnetism and Magnetic Materials, vol. 355, pp. 22-30, 2014.
31. D. Zhao, X. Wang, X, Zeng, Q. Xia, J. Tang. “Preparation and inductive heating property of Fe3O4-chitosan composite nanoparticles in an AC magnetic field for localized hyperthermia”. Journal of Alloys and Compounds, vol. 477, pp. 739-743, 2009.
32. H. O. Park, K. Im, S. Lee, D. Kim, D. Lee, Y. Lee, K. Kim, K. Kim. “Preparation and characterization of magnetic chitosan particles for hyperthermia application”. Journal of Magnetism and Magnetic Materials, vol. 293, pp. 328-333, 2005.
33. C. Sanjai, S. Kothan, P. Gonil, S. Saesoo, W. Sajomsang. “Chitosan-triphosphate nanoparticles for encapsulation of super-paramagnetic iron oxide as an MRI contrast agent”. Carbohydrate Polymers, vol. 104, pp. 231-237, 2014.
34. Z. Tsai, J. Wang, H. Kuo, C. Shen, J. Wang, T. Yen. “In situ preparation of high relaxivity iron oxide nanoparticles by coating with chitosan: A potential MRI contrast agent useful for cell tracking”. Journal of Magnetism and Magnetic Materials, vol. 322, pp. 208-213, 2010.
35. Y. Huang, B. Cao, X. Yang, Q. Zhang, X. Han, Z. Guo. “Gd complexes of diethylenetriaminepentaacetic acid conjugates of low-molecular-weight chitosan oligosaccharide as a new liver-specific MRI contrast agent”. Magnetic Resonance Imaging, vol. 31, pp. 604-609, 2013.
36. T. Ahmad, H Bae, Y. Iqbal, I. Rhee, S. Hong, Y. Chang, J. Lee, D. Sohn. “Chitosan-coated nickel-ferrite nanoparticles as contrast agents in magnetic resonance imaging”. Journal of Magnetism and Magnetic Materials, vol. 381, pp. 151-157, 2015.
37. M. Schafer, T. R. Schneider, G. M. Scheldrick. (December, 1996). Crystal structure of vancomycin. Structure, 4(12), pp. 1509-1515.
38. M. E. Roy, M. P. Peppers, L. A. Whiteside, R. M. LaZear. (March, 2014). Vancomycin concentration in synovial fluid: direct infection into the knee vs intravenous infusion. The Journal of Arthroplasty, 29(3), pp. 564-568.
39. V. P. Mantripragada, A. C. Jayasuriya. (October, 2016). Effect of dual delivery of antibiotics (vancomycin and cefazolin) and BMP-7 from chitosan microparticles on Staphylococcus epidermis and pre-osteoblasts in vitro. Materials Science and Engineering C, 67, pp. 409-417.
40. T. Cerchiara et al. (June, 2016). Microparticles based on chitosan/carboxymethylcellulose polyelectrolyte complexes for colon delivery of vancomycin. Carbohydrate polymers. 143, pp. 124-130.
41. Z. Yang, J. Liu, J. Gao, S. Chen, G. Huang. (November, 2015). Chitosan coated vancomycin hydrochloride liposomes: characterizations and evaluation. International Journal of Pharmaceutics. 495, pp. 508-515.
42. Y. S. Kang, S. Risbud, J. F. Rabolt, P. Stroeve. (1996). Synthesis and Characterization of Nanometer-Size Fe3O4 and γ-Fe2O3. Chem. Mater. 8, pp. 2209-2211.
43. H. Li, L. Qin, Y. Feng, L. Hu, C. Zhou. (January, 2015). Preparation and characterization of highly water-soluble magnetic Fe3O4 nanoparticles via surface double-layered self-assembly method of sodium alpha-olefin sulfonate. Journal of Magnetism and Magnetic Materials. 384(15), pp. 213-218.
44. H. Lv, R. Jiang, Y. Li, X. Zhang, J. Wang. (March, 2015). Microemulsion-mediated hydrothermal growth of pagoda-like Fe3O4 microstructures and their application in a lithium-air battery. Ceramics International. 41(7), pp. 8843-8848.

Y45. S. Kang, S. Risbud, J. F. Rabolt, P. Stroeve. (1996). Synthesis and Characterization of Nanometer-Size Fe3O4 and γ-Fe2O3. Chem. Mater. 8, pp. 2209-2211

What is claimed is:

1. A microbead comprising cross-linked chitosan, a magnetic nanoparticle, and an agent.

2. The microbead of claim 1, wherein the chitosan is cross-linked to a polymer.

3. The microbead of claim 3, wherein the polymer is polyethylene dimethacrylate (PEGDMA).

4. The microbead of claim 1, wherein the microbead comprises an effective amount of an agent selected from the group consisting of a polypeptide, polynucleotide or small compound.

5. The microbead of claim 1, wherein the agent is an analgesic, angiogenic agent, antimicrobial, antibody, antifungal, anti-inflammatory, anti-thrombotic, chemotherapeutic, growth factor, hormone, or steroid agent.

6. The microbead of claim 1, wherein the effective amount of the agent is sufficient to reduce the survival or proliferation of a fungal or bacterial cell.

7. The microbead of claim 1, wherein the fungal cell is Candida albicans and/or the bacterial cell is Pseudomonas aeruginosa (lux) or Staphylococcus aureus.

8. The microbead of claim 1, wherein the composition releases at least about 0.2-50 μg of an antimicrobial agent per hour.

9. The microbead of claim 1, wherein the microbead is biodegradable over at least about one, two, three, four, or five days, or one, two, three, or four weeks.

10. A method for producing a chitosan microbead, the method comprising:
(a) dissolving chitosan in an acidic solution;
(b) adding magnetic nanoparticles and an agent to the solution;
(c) providing a mixture of surfactant, oil, and a polymer; and
(d) adding the chitosan solution of step (a) to the oil and incubating until beads form.

11. The method of claim 1, wherein step (a) further comprises incorporating an effective amount of one or more agents into the solution.

12. A microbead generated according to the method of claim 11.

13. A method for treating or preventing an infection in a subject at a site of trauma, the method comprising contacting the site with a chitosan microbead of any one of claims 1-9 and applying an external stimulus.

14. The method of claim 13, wherein the trauma is selected from the group consisting of a fracture, open fracture, wound, complex wound, and surgical site.

15. The method of claim 13, wherein the agent is selected from the group consisting of an analgesic, angiogenic agent, antimicrobial, antibody, antifungal, anti-inflammatory, anti-thrombotic, chemotherapeutic, growth factor, hormone, or steroid agent.

16. The method of claim 14, wherein the antimicrobial agent is selected from the group consisting of antifungal, antibacterial, and antiviral agents.

17. The method of claim 14, wherein the antimicrobial agents are amphotericin B, vancomycin, and/or amikacin.

18. The method of claim 14, wherein the effective amount of the agent is sufficient to reduce the survival or proliferation of a bacterial cell.

19. The method of claim 14, wherein the composition releases at least about 0.2-50 μg of an antimicrobial agent per hour.

20. The method of claim 14, wherein the method reduces fungi or bacteria present at the site by at least about 20-100% at 72 hours after contact with the chitosan-microbead composition relative to an untreated control site.

21. The method of claim 13, wherein the external stimulus is a magnetic field.

22. A method for the local and temporally controlled delivery of an agent to a site, the method comprising contacting the site with a chitosan microbead comprising an agent and applying an external stimulus at a desired time point, thereby temporally controlling delivery of the agent to the site.

23. The method of claim 22, wherein the agent is selected from the group consisting of an analgesic, angiogenic agent, antimicrobial, antibody, antifungal, anti-inflammatory, anti-thrombotic, chemotherapeutic, growth factor, hormone, or steroid agent.

24. The method of claim 22, wherein the microbead releases about 2 μg-1000 mg of the agent in 1-72 hours.

25. The method of claim 22, wherein the stimulus is a magnetic field.

26. The method of claim 22, wherein the stimulus is applied for 30 minutes.

27. A kit comprising a chitosan microbead of claim 1 for use in treating a trauma site or delivering an agent.

28. The kit of claim 24, wherein the chitosan microbead comprises an agent selected from the group consisting of an analgesic, angiogenic agent, antimicrobial, antibody, antifungal, anti-inflammatory, anti-thrombotic, chemotherapeutic, growth factor, hormone, or steroid agent.
